(19) United States
(12) Patent Application Publication
(10) Pub. No.: US 2008/0213786 A1
(43) Pub. Date: Sep. 4, 2008

(54) Treatment of rheumatoid arthritis with galectin-3 antagonists

(75) Inventors: Vincent Jacques Hurez, Albany, CA (US); Seth G. Michelson, San Jose, CA (US); Herbert Struemper, Bethlehem, PA (US); Leif Gustaf Wennerberg, Mountain View, CA (US)

Correspondence Address: ENTELOS, INC.;c/o Law Offices of Karen E. Flick, P.O. Box 515, El Granada, CA, 94018-0515, US

(73) Assignee: Entelos, Inc., Foster City, CA (US)

(21) Appl. No.: 12/082,508

(22) Filed: Apr. 11, 2008

Related U.S. Application Data
(62) Division of application No. 11/017,213, filed on Dec. 17, 2004, now abandoned.
(60) Provisional application No. 60/530,765, filed on Dec. 17, 2003.

(51) Int. Cl. C12Q 1/68; C12Q 1/02; G01N 33/53; G01N 33/00

(52) U.S. Cl. 435/6; 435/29; 435/7.24; 436/86

(57) ABSTRACT

The invention encompasses a novel method of treating an inflammatory disease, such as rheumatoid arthritis, and novel methods of identifying and screening for drugs useful in the treatment of inflammatory diseases, such as rheumatoid arthritis, and their clinical symptoms. The inventors have made the discovery that the activity of galectin-3, a β-galactoside-binding lectin known to have an effect on some cancers, has a significant impact on the pathophysiology of rheumatoid arthritis. The symptoms of an inflammatory disease, such as rheumatoid arthritis, may be alleviated by administering a compound that inhibits the activity of galectin-3.

### A. RELATED APPLICATIONS

This application claims the benefit of U.S. Provisional Application No. 60/530,765, filed Dec. 17, 2003, which is herein incorporated by reference.

### B. FIELD OF THE INVENTION

This invention relates to novel methods of treating rheumatoid arthritis and methods of identifying compounds useful in treating rheumatoid arthritis.

### C. BACKGROUND OF THE INVENTION

There are more than 100 forms of arthritis and of them, rheumatoid arthritis is the most painful and crippling form. Rheumatoid arthritis, a common disease of the joints, is an autoimmune disease that affects over 2 million Americans, with a significantly higher occurrence among women than men. In rheumatoid arthritis, the membranes or tissues (synovial membranes) lining the joints become inflamed (synovitis). Over time, the inflammation may destroy the joint tissues, leading to disability. Because rheumatoid arthritis can affect multiple organs of the body, rheumatoid arthritis is referred to as a systemic illness and is sometimes called rheumatoid disease. The onset of rheumatoid disease is usually in middle age, but frequently occurs in one's 20s and 30s. See the Merck Manual, Sixteenth Edition, section 106 for a further discussion.

The pain and whole-body (systemic) symptoms associated with rheumatoid disease can be disabling. Over time, rheumatoid arthritis can cause significant joint destruction, leading to deformity and difficulty with daily activities. It is not uncommon for people with rheumatoid arthritis to suffer from some degree of depression, which may be caused by pain and progressive disability. A study reports that one-fourth of people with rheumatoid arthritis are unable to work by 6 to 7 years after their diagnosis, and half are not able to work after 20 years (O'Dell J R (2001). Rheumatoid arthritis: The clinical picture. In W J Koopman, ed., Arthritis and Allied Conditions: A Textbook of Rheumatology, 14th ed., vol. 1, chap. 58, pp. 1153-1186. Philadelphia: Lippincott Williams and Wilkins). Musculoskeletal conditions such as rheumatoid arthritis cost the U.S. economy nearly $65 billion per year in medical care and indirect expenses such as lost wages and production.

Synovial inflammation, rapid degradation of cartilage, and erosion of bone in affected joints are characteristic of rheumatoid arthritis (RA). Recent evidence indicates that skeletal tissue degradation and inflammation are regulated through overlapping but not identical biological processes in the rheumatoid joint and that therapeutic effects on these two aspects need not be correlated. Due to the complexity of the biological processes in the joint, mathematical and computer models can be used to help better understand the interactions between the various tissue compartments, cell types, mediators, and other factors involved in joint disease and healthy homeostasis. Several researchers have constructed simple models of the mechanical environment of the joint, rather than the biological processes of rheumatoid arthritis, and compared the results to patterns of disease and development in cartilage and bone (Wynarsky & Greenwald, J. Biomech., 16:241-251, 1983; Pollatschek & Nahir, J. Theor. Biol., 143:497-505, 1990; Beaupre et al., J. Rehabil. Res. Dev., 37:145-151, 2000; Shi et al., Acta Med. Okayama, 17:646-653, 1999). A computer manipulable mathematical model of joint diseases that includes multiple compartments including the synovial membrane and the interactions of these compartments is described in published PCT application WO 02/097706, published 5 Dec. 2002 and U.S. patent application Ser. No. 10/154,123, published 24 Apr. 2003 as 2003-0078759. Both publications are incorporated herein by reference in their entirety.

Rheumatoid arthritis is a chronic disease that, at present, can be controlled but not cured. The goal of treatment is relief of symptoms and keeping the disease from getting worse. The goals of most treatments for rheumatoid arthritis are to relieve pain, reduce inflammation, slow or stop the progression of joint damage, and improve a person's ability to function. Current approaches to treatment include lifestyle changes, medication, surgery, and routine monitoring and care. Medications used for the treatment of rheumatoid arthritis can be divided into two groups based on how they affect the progression of the disease: (1) symptom-relieving drugs and (2) disease-modifying drugs.

Medications to relieve symptoms, such as pain, stiffness, and swelling, may be used. Nonsteroidal anti-inflammatory drugs (NSAIDs), such as aspirin, ibuprofen, and naproxen are used to control pain and may help reduce inflammation. They do not control the disease or stop the disease from getting worse. Corticosteroids, such as prednisone and methylprednisone (Medrol), are used to control pain and reduce inflammation. They may control the disease or stop the disease from getting worse; however, using corticosteroids as the only therapy for an extended time is not considered the best treatment. Corticosteroids are often used to control symptoms and flares of joint inflammation until anti-rheumatic drugs reach their full effectiveness, which can take up to 6 months. Nonprescription medications such as acetaminophen and topical medications such as capsaicin are used to control pain, but do not usually affect joint swelling or worsening of the disease.

Disease-modifying anti-rheumatic drugs (DMARDs) are used to control the progression of rheumatoid arthritis and to try to prevent joint deterioration and disability. These anti-rheumatic drugs are often given in combination with other anti-rheumatic drugs or with other medications, such as nonsteroidal anti-inflammatory drugs. Disease-modifying anti-rheumatic drugs commonly prescribed for rheumatoid arthritis include antimalarial medications such as hydroxychloroquine (Plaquenil) or chloroquine (Aralen), methotrexate (e.g., Rheumatrex), sulfasalazine (Azulfidine), leflunomide (Arava), etanercept (Enbrel), infliximab (Remicade), adalimumab (Humira) and anakinra (Kineret). DMARDs less commonly prescribed for rheumatoid arthritis include azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). Some of these anti-rheumatic drugs can take up to 6 months to work. Many have serious side effects.

Thus a need exists for new, therapeutically effective drugs for the treatment of rheumatoid arthritis as well as new methods for identifying such drugs.

### D. SUMMARY OF THE INVENTION

In one aspect, the invention provides methods for alleviating at least one symptom of rheumatoid arthritis comprising administering a therapeutically effective amount of an antagonist of galectin-3 activity to a patient having rheumatoid arthritis. In a preferred embodiment, the antagonist decreases the galectin-3 activity by at least 35%, more preferably by at least 60% and most preferably by at least 95%. The antagonist of galectin-3 activity may be a protein, nucleic acid, carbohydrate or small molecule inhibitor. A “small molecule” is defined herein as a molecule having a molecular weight of less than 1000 daltons. Preferred antagonists include, but are not limited to, pectins, lactose, N-acetyllactosamines and thiogalactoside derivatives. Preferred antibody antagonists include B2C10, 9C4 and M3/38. In one embodiment, the method contemplates treating rheumatoid arthritis in patients who are resistant to traditional methotrexate therapy. In preferred embodiments, the patient is a methotrexate resistant patient, a TNF-α blockade cartilage nonresponder (CNR), a TNF-α blockade hyperplasia nonresponder (HNR), or a TNF-α blockade double nonresponder (DNR).

In another aspect, the invention provides methods for decreasing density of synovial cells in a joint comprising administering a therapeutically effective amount of an antagonist of galectin-3 activity to a patient having a condition associated with abnormally increased synovial cell density. In a preferred embodiment, the antagonist decreases the galectin-3 activity by at least 35%, more preferably by at least 60% and most preferably by at least 95%.

The invention also provides methods for decreasing cartilage degradation in a joint comprising administering a therapeutically-effective amount of an antagonist of galectin-3 activity to a patient having a condition associated with an abnormally high rate of cartilage degradation. In a preferred embodiment, the antagonist decreases the galectin-3 activity by at least 35%, more preferably by at least 60% and most preferably by at least 95%.

Yet another aspect of the invention provides methods for decreasing IL-6 concentration in synovial tissue comprising administering a therapeutically effective amount of an antagonist of galectin-3 activity to a patient having a condition associated with an abnormally high concentration of IL-6 in synovial tissue. In a preferred embodiment, the antagonist decreases the galectin-3 activity by at least 35%, more preferably by at least 60% and most preferably by at least 95%.

In another aspect, the invention provides methods of alleviating at least one symptom of an inflammatory disease comprising administering a therapeutically effective amount of an antagonist of galectin-3 activity to a patient suffering from an inflammatory disease. In a preferred embodiment, the antagonist decreases the galectin-3 activity by at least 35%, more preferably by at least 60% and most preferably by at least 95%. In preferred embodiments, the inflammatory disease is selected from the group consisting of diabetes, arteriosclerosis, inflammatory aortic aneurysm, restenosis, ischemia/reperfusion injury, glomerulonephritis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. More preferably the inflammatory disease is an arthritis, such as rheumatoid arthritis, psoratic arthritis, coxarthritis, osteoarthritis, or polyarthritis. Most preferably, the inflammatory disease is rheumatoid arthritis.

Yet another aspect of the invention provides methods of alleviating at least one symptom of rheumatoid arthritis, comprising administering an antagonist of galectin-3 activity and an anti-rheumatic drug to a patient having rheumatoid arthritis. The anti-rheumatic drug can be any drug that, in combination with galectin-3 antagonism, provides a better clinical outcome than treatment with galectin-3 antagonism or the anti-rheumatic drug alone. The anti-rheumatic drug can be a symptom-relieving anti-rheumatic drug or a disease-modifying anti-rheumatic drug. Exemplary symptom-relieving anti-rheumatic drugs include aspirin, ibuprofen, naproxen, and corticosteroids, such as prednisone and methylprednisolone (Medrol). Exemplary disease-modifying anti-rheumatic drugs include hydroxycholoroquine (Plaquenil), chloroquine (Aralen), methotrexate (e.g., Rheumatrex), sulfasalazine (Azulfidine), leflunomide (Arava), etanercept (Enbrel), infliximab (Remicade), adalimumab (Humira), anakinra (Kineret), azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). In preferred embodiments, the anti-rheumatic drug is methotrexate, a TNF-α antagonist, such as Etanercept, an interleukin-1 receptor antagonist, such as Anakinra, or a steroid, such as methylprednisolone.

One aspect of the invention provides methods for manufacturing a drug for use in the treatment of rheumatoid arthritis comprising (a) identifying a compound as useful in the treatment of rheumatoid arthritis and (b) formulating said compound for human consumption.

The compound is identified by (i) comparing an amount of galectin-3 activity in the presence of the compound with an amount of galectin-3 activity in the absence of the compound; and (ii) identifying the compound as useful in the treatment of rheumatoid arthritis when the amount of galectin-3 activity in the presence of the compound is lower than the amount of galectin-3 activity in the absence of the compound. Preferably, the compound is selected as useful in the treatment of rheumatoid arthritis when the amount of galectin-3 activity is at least 35% lower in the presence of the compound than in the absence of the compound. More preferably, the galectin-3 activity will be at least 60% lower and most preferably 95% lower in the presence of the compound.

Yet another aspect of the invention also provides methods for identifying a compound useful in the treatment of rheumatoid arthritis, which method comprises (a) comparing an amount of galectin-3 activity in the presence of the compound with an amount of galectin-3 activity in the absence of the compound; and (b) selecting the compound as useful in the treatment of rheumatoid arthritis when the amount of galectin-3 activity in the presence of the compound is lower than the amount of galectin-3 activity in the absence of the compound. In one embodiment, a collection of compounds may be screened by repeating steps (a) and (b) for each compound in a collection of compounds, wherein at least one compound of the collection is selected as useful for the treatment of rheumatoid arthritis.

The amount of galectin-3 activity can be determined by a variety of methods. One method for measuring galectin-3 activity comprises observing an amount of leukocyte apoptosis in the presence of the compound that is higher than an amount of leukocyte apoptosis in the absence of the compound. Preferably, the compound useful for the treatment of rheumatoid arthritis will cause an increase in macrophage apoptosis. In a preferred embodiment of the invention, the compound is identified or selected as useful in the treatment of rheumatoid arthritis when the amount of macrophage apoptosis in the presence of the compound is at least 50% greater than the amount of macrophage apoptosis in the absence of the compound. More preferably the compound will increase macrophage apoptosis by at least 100% and most preferably by at least 200%.

The amount of macrophage apoptosis may be determined by any apoptosis measurement technique, now known or discovered in the future. One embodiment of the invention measures the amount of macrophage apoptosis by a process comprising the steps of exposing a population of cells to an inducer of apoptosis in the presence or absence of the compound, and measuring the percentage of cells having DNA fragmentation, wherein the percentage of cells having DNA fragmentation represents the amount of macrophage apoptosis. The percentage of cells having DNA fragmentation may be measured by any method know in the art, including propidium iodide uptake or TUNEL assay (terminal deoxynucleotidyl transferase-mediated 2′-deoxyuridine 5′-triphosphate-biotin nick-end labeling). In yet another embodiment of the invention, the amount of macrophage apoptosis is measured by a process comprising the steps of exposing a population of cells to an inducer of apoptosis in the presence or absence of the compound, and measuring the percentage of cells expressing phosphatidylserine on the extracellular surface of the cell membrane, wherein the percentage of cells expressing phosphatidylserine on the extracellular surface of the cell membrane represents the amount of macrophage apoptosis. Preferably the expression of phosphatidylserine on the extracellular surface of the cytoplasmic membrane is measured by binding of annexin V to the phosphatidylserine. Preferred inducers of apoptosis include, but are not limited to, sFas ligand, anti-Fas or TRAIL or hypoxia.

Yet another method for measuring the amount of galectin-3 activity comprises (a) comparing an amount of leukocytes that migrate through at least one layer of endothelial cells in the presence of the compound with an amount of leukocytes that migrate through at least one layer of endothelial cells in the absence of the compound; and (b) identifying the compound as an antagonist of galectin-3 activity when the amount of migrating leukocytes in the presence of the compound is less than the amount of migrating leukocytes in the absence of the compound. Preferably, the amount of leukocytes that migrate through the endothelial layer(s) will be at least 35% lower in the presence of the compound than in the absence of the compound. More preferably, the amount of migrating leukocytes will be at least 60% lower and most preferably at least 95% lower in the presence of the compound. In a preferred embodiment, the leukocytes are monocytes.

The amount of galectin-3 activity can also be determined by the process comprising observing an amount of a cytokine produced by a first population of macrophages in the presence of the compound that is lower than an amount of the cytokine produced by a second population of macrophages in the absence of the compound. The cytokine preferably is tumor necrosis factor-α (TNF-α) or interleukin-1 (IL-1). In a preferred embodiment, the amount of cytokine produced by the first population of macrophages in the presence of the compound is at least 40% lower than the amount of cytokine produced by the second population in the absence of the compound. More preferably the amount is at least 60% lower and most preferably at least 80% lower in the absence of the compound.

Another aspect of the invention provides packages comprising an antagonist of galectin-3 activity and a label with instructions for administering the antagonist for treating rheumatoid arthritis.

### BRIEF DESCRIPTION OF THE FIGURES

For a better understanding of the nature and objects of some embodiments of the invention, reference should be made to the following detailed description taken in conjunction with the accompanying drawings, in which:

FIG. 1 demonstrates the effect of galectin-3 blockade on synovial cell density in a typical rheumatoid arthritis patient.

FIG. 2 demonstrates the effect of galectin-3 blockade on the rate of cartilage degradation in a typical rheumatoid arthritis patient.

FIG. 3 demonstrates the effect of galectin-3 blockade on IL-6 in synovial tissue in a typical rheumatoid arthritis patient.

FIG. 4 demonstrates simulation of galectin-3 blockade on individual significant biological processes in a typical rheumatoid arthritis patient.

FIG. 5 demonstrates simulation of turning off galectin-3 blockade in individual significant biological processes in a typical rheumatoid arthritis patient.

FIG. 6 demonstrates simulation of galectin-3 blockade on individual significant biological processes in a methotrexate resistant patient.

FIG. 7 demonstrates the effect of galectin-3 blockade on synovial cell density in a methotrexate resistant patient.

FIG. 8 demonstrates the effect of galectin-3 blockade on the rate of cartilage degradation in a methotrexate resistant patient.

FIG. 9 demonstrates the effect of galectin-3 blockade on IL-6 in synovial tissue in a methotrexate resistant patient.

FIG. 10 provides a comparison of galectin-3 inhibition with expected increase of macrophage and T cell apoptosis and decreased monocyte recruitment and cytokine production rates at the ‘upper maximum effect’ of galectin-3 antagonism.

FIG. 11 provides a comparison of galectin-3 inhibition with expected increase of macrophage and T cell apoptosis and decreased monocyte recruitment and cytokine production rates at the ‘most likely maximum effect’ of galectin-3 antagonism.

FIG. 12 illustrates the induction of chemotaxis of monocytes from healthy donors in response to galectin-3 alone or in the presence of an anti-galectin-3 antibody, B2C10.

FIG. 13 illustrates the induction of chemotaxis of monocytes from rheumatoid arthritis patient in response to galectin-3 alone or in the presence of an anti-galectin-3 antibody, B2C10.

FIG. 14 illustrates induction of chemotaxis of monocytes from a single healthy donor by galectin-3 in combination with chemotactic cytokines, (A) N-formyl-met-leu-phe (FMPL); (B) Stromal cell-Derived Factor-1 (SDF-1) and (C) Monocyte Chemotactic Protein-1 (MCP-1).

FIG. 15 illustrates the effect of galectin-3 blockade on fMLP-induced chemotaxis.

FIG. 16A illustrates the results of galectin-3 blockade across a panel of monocytes obtained from different rheumatoid arthritis patients. FIG. 16B illustrates the results of galectin-3 blockade across a panel of synovial fluid obtained from different rheumatoid arthritis patients.

FIG. 17 illustrates the effect of inhibition of galectin-3 activity on monocyte chemotaxis induced by synovial fluid from rheumatoid arthritis patients.

### DETAILED DESCRIPTION

### A. Overview

In general this invention can be viewed as encompassing novel methods of treating an inflammatory disease, such as rheumatoid arthritis, and novel methods of identifying and screening for drugs useful in the treatment of inflammatory diseases, such as rheumatoid arthritis, and their clinical symptoms. The inventors have made the discovery that the activity of galectin-3, a β-galactoside-binding lectin known to have an effect on some cancers, has a significant impact on the pathophysiology of rheumatoid arthritis. The symptoms of an inflammatory disease, such as rheumatoid arthritis, may be alleviated by administering a compound that inhibits the activity of galectin-3.

### B. Definitions

The term “abnormally high concentration of IL-6 in synovial tissue,” as used herein, refers to a level of IL-6 in the synovial tissue of the diseased joint that is at least 3 standard deviations higher than that found in a normal, non-diseased, joint.

The term “abnormally high rate of cartilage degradation,” as used herein, refers to a detectable joint space narrowing as determined by standard radiographic measures. In a non-diseased joint narrowing is not detectable.

The term “abnormally increased synovial cell density,” as used herein, refers to a condition in which the synovial tissue of a joint contains a number of synovial cells that is at least ten-times higher than the number of synovial cells found in the synovial tissue of a normal, i.e., non-diseased, joint.

“Administering” means any of the standard methods of administering a pharmaceutical composition known to those skilled in the art. Examples include, but are not limited to intravenous, intramuscular or intraperitoneal administration.

The term “antagonist of galectin-3 activity,” as used herein, refers to the property of inhibiting any one of the three biological activities of galectin-3 shown to be relevant to rheumatoid arthritis: (1) monocyte and T-cell recruitment, (2) monocyte/macrophage and T-cell apoptosis, and (3) macrophage cytokine production. Inhibition need not be 100% effective in order to be antagonistic.

The term “drug” refers to a compound of any degree of complexity that can affect a biological system, whether by known or unknown biological mechanisms, and whether or not used therapeutically. Examples of drugs include typical small molecules (molecules having molecular weights of less than 1000 daltons) of research or therapeutic interest; naturally-occurring factors such as endocrine, paracrine, or autocrine factors, antibodies, or factors interacting with cell receptors of any type; intracellular factors such as elements of intracellular signaling pathways; factors isolated from other natural sources; pesticides; herbicides; and insecticides. Drugs can also include, agents used in gene therapy such as DNA and RNA. Also, antibodies, viruses, bacteria, and bioactive agents produced by bacteria and viruses (e.g., toxins) can be considered as drugs. A response to a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. In some instances, drugs can exert their effects by interacting with a protein. For certain applications, drugs can also include, for example, compositions including more than one drug or compositions including one or more drugs and one or more excipients.

“Inflammatory diseases” refers to a class of diverse diseases and disorders that are characterized by any one of the following: the triggering of an inflammatory response; an upregulation of any member of the inflammatory cascade; the downregulation of any member of the inflammatory cascade. Inflammatory diseases include diabetes, arteriosclerosis, inflammatory aortic aneurysm, restenosis, ischemia/reperfusion injury, glomerulonephritis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. The singular term “inflammatory disease” includes any one or more diseases selected from the class of inflammatory diseases, and includes any compound or complex disease state wherein a component of the disease state includes a disease selected from the class of inflammatory diseases.

The term “joint,” as used herein, comprises the synovial tissue, synovial fluid, articular cartilage, bone tissues, and their cellular and extracellular composition, and the soluble mediators they contain.

The term “methotrexate resistant patient” refers to a rheumatoid arthritis patient who does not effectively respond to methotrexate treatment or who initially responds to methotrexate and becomes refractory over time.

The term “patient” refers to any warm-blooded animal, preferably a human. Patients having rheumatoid arthritis can include, for example, patients that have been diagnosed with rheumatoid arthritis, patients that exhibit one or more of the symptoms associated with rheumatoid arthritis, or patients that are progressing towards or are at risk of developing rheumatoid arthritis.

As used herein, a “therapeutically effective amount” of a drug of the present invention is intended to mean that amount of the compound that will inhibit an increase in synovial cells in a rheumatic joint or decrease the rate of cartilage degradation in a rheumatic joint or decrease IL-6 concentration in synovial tissue, and thereby cause the regression and palliation of the pain and inflammation associated with rheumatoid arthritis

The term “TNF-α blockade resistant patient” refers to a rheumatoid arthritis patient who does not effective respond to TNF-α blockade or who initially responds to TNF-α blockade and becomes refractory over time.

The term “TNF-α blockade cartilage nonresponder” refers to a rheumatoid arthritis patient with low initial TNF-α activity who shows decreased synovial hyperplasia, but minimal reduction in cartilage degradation in response to TNF-α blockade.

The term “TNF-α blockade hyperplasia nonresponder” refers to a rheumatoid arthritis patient with abnormally high or resistant levels of TNF-α activity who yields improvement in cartilage degradation but little decrease in synovial hyperplasia in response to TNF-α blockade.

The term “TNF-α blockade double nonresponder” refers to a rheumatoid arthritis patient with negligible initial TNF-α activity who shows poor response in both synovial hyperplasia and cartilage degradation in response to TNF-α blockade.

### C. Identifying a Compound Useful in Treating Rheumatoid Arthritis

One aspect of the invention is a method of identifying a compound useful in the treatment of rheumatoid arthritis, which method comprises (a) comparing an amount of galectin-3 activity in the presence of the compound with an amount galectin-3 activity in the absence of the compound; and (b) selecting the compound as useful in the treatment of rheumatoid arthritis when the amount of galectin-3 activity in the presence of the compound is lower than the amount of galectin-3 activity in the absence of the compound. The dynamic processes related to the biological state of a human joint afflicted with rheumatoid arthritis involve various biological variables related to the processes involved in cartilage metabolism, tissue inflammation, and tissue hyperplasia, including the following:
macrophage population dynamics including: recruitment, activation, proliferation, apoptosis and their regulation,
T cell population dynamics including: recruitment, antigen-dependent and antigen-independent activation, proliferation, apoptosis and their regulation
Fibroblast-like synoviocyte (FLS) population dynamic including: influx in the tissue, proliferation, and apoptosis and their regulation
chondrocyte population dynamics including: proliferation and apoptosis
synthesis and regulation of a variety of proteins, including: growth factors, cytokines, chemokines, proteolytic enzymes and matrix proteins, by the different cell type represented (macrophages, FLS, T cells, chondrocytes).
expression of adhesion molecules by endothelial cells
transport of mediators between synovial tissue and cartilage
interaction between cytokines or proteases and their natural inhibitors, antigen presentation, and

binding of therapeutic agents to cellular mediators (anti-TNF-α agents etanercept and infliximab and IL-1 RA antagonist anakinra).

Based on observations of an in silico model providing mathematical representations of a human joint afflicted with rheumatoid arthritis, we found that antagonists of galectin-3 will alleviate the symptoms of rheumatoid arthritis, especially decreasing the density of synovial cells, decreasing the rate of cartilage degradation, and decreasing the concentration of IL-6 in synovial tissue. These observations also take into account vascular volume and the effect of therapeutic agents such as methotrexate, steroids, non-steroidal anti-inflammatory drugs, soluble TNF-α receptor, TNF-α antibody, and interleukin-1 receptor antagonists.

In silico modeling integrates relevant biological data—genomic, proteomic, and physiological—into a computer-based platform to reproduce a system's control principles. Given a set of initial conditions representing a defined disease state, these computer-based models can simulate the system's future biological behavior, a process termed biosimulation.

Using a “top-down” approach that starts by defining a general set of behaviors indicative of rheumatoid arthritis, these behaviors are used as constraints on the system. A set of nested subsystems is developed to define the next level of underlying detail. For example, given a behavior such as cartilage degradation in rheumatoid arthritis, the specific mechanisms inducing that behavior are each modeled in turn, yielding a set of subsystems, which themselves are deconstructed and modeled in detail. The control and context of these subsystems is, therefore, already defined by the behaviors that characterize the dynamics of the system as a whole. The deconstruction process continues modeling more and more biology, from the top down, until there is enough detail to replicate the known biological behavior of rheumatoid arthritis.

When using a top-down approach, data is identified and collected to support two specific purposes: (1) describing basic biology and (2) describing physiological function or behavior of the whole system. Data describing physiological functions or behavior of the whole system are selected early in the development of the model. These data represent the broad range of behaviors of the models system, i.e. cartilage degradation as a measurement (behavior) of rheumatoid arthritis patients. These data are human in vivo data based on well-established clinical trials. Data describing basic biology is selected to sufficiently model the subsystems required to simulate the selected behaviors. These data can be human or animal (where human is preferred but not always available) in vivo, in vitro, or ex vivo data which provide an understanding of the underlying biology.

The top-down approach was used to develop a model of rheumatoid arthritis in a human joint. A similar model is described in co-pending U.S. patent application Ser. No. 10/154,123, published 24 Apr. 2003 as 2003-0078759. Three key clinical outcomes are of particular interest in the present model: synovial cell density, the rate of cartilage degradation and the level of IL-6 in synovial tissue. Rheumatoid arthritis is a systemic inflammatory disease with elevated levels of proinflammatory cytokines in peripheral blood, especially IL-6. C-reactive protein (CRP) is a common marker of inflammation which is routinely measured in the plasma, and several studies have shown a correlation between the concentration of IL-6 and the concentration of CRP in rheumatoid arthritis patients. Therefore, IL-6 concentration in either the joint or the plasma represents a good marker of inflammation. The chronic inflammation associated with rheumatoid arthritis leads to synovial cell hyperplasia and ultimately significant cartilage degradation in rheumatic joints.

The explicit representation of the underlying biology of the disease allows the modulation of each subsystem alone or in combination to identify the one(s) with most impact on a specific clinical outcome, such as cartilage degradation rate or synovial cell density. By focusing modeling and data collection efforts on those subsystems with the greatest impact on the phenotypic onset and progression or rheumatoid arthritis, this approach can help more clearly represent the system's complexity and identify causal factors underlying the pathophysiology of rheumatoid arthritis. By modulating, in silico, each subsystem (e.g. knocking-out one cell type or intercellular mediator, or blocking one particular biological process), its contribution to the overall disease pathophysiology can be evaluated to better understand the biological phenomena driving rheumatoid arthritis, thus identifying the best and most relevant targets.

In the case of rheumatoid arthritis, the disease state can be represented as outputs associated with, for example, enzyme activities, product formation dynamics, and cellular functions that can indicate one or more biological processes that cause, affect, or are modified by the disease state. Typically, the outputs of the computer model include a set of values that represent levels or activities of biological constituents or any other behavior of the disease state. Based on these outputs, one or more biological processes can be designated as critical biological processes.

The computer model can be executed to represent a modification to one or more biological processes, as described in greater detail in co-pending application, U.S. Ser. No. 10/938,072 filed Sep. 10, 2004. In particular, a modification to a biological process can be represented in the computer model to identify the degree of connection (e.g., the degree of correlation) between the biological process and rheumatoid arthritis. For example, a modification to a biological process can be represented in the computer model to identify the degree to which the biological process causes, affects, or is modified by rheumatoid arthritis. A biological process can be identified as causing rheumatoid arthritis if a modification to this biological process is observed to produce symptoms associated with rheumatoid arthritis, i.e., increased synovial cell density, cartilage degradation rate, bone erosion and IL-6 levels in the synovial tissue. In some instances, a modification to a biological process can be represented in the computer model to identify the degree of connection between other biological processes and rheumatoid arthritis.

In some instances, identifying the set of biological processes can include sensitivity analysis. Sensitivity analysis can involve prioritization of biological processes that are associated with the disease state and can be performed with different configurations of the computer model to determine the robustness of the prioritization. In some instances, sensitivity analysis can involve a rank ordering of biological processes based on their degree of connection to the disease state. Sensitivity analysis allows a user to determine the importance of a biological process in the context of the disease state. An example of a biological process of greater importance is a biological process that increases the severity of the disease state. Thus, inhibiting this biological process can decrease the severity of the disease state. The importance of a biological process can depend not only on the existence of a connection between that biological process and the disease state but also on the extent to which that biological process has to be modified to achieve a change in the severity of the disease state. In a rank ordering, a biological process that plays a more important role in the disease state typically gets a higher rank. The rank ordering can also be done in a reverse manner, such that a biological process that plays a more important role in the disease state gets a lower rank. Typically, the set of biological processes include biological processes that are identified as playing a more important role in the disease state.

During the process of sensitivity analysis of rheumatoid arthritis the activity of biological processes such as to monocyte recruitment, T-cell recruitment, leukocyte apoptosis, and cytokine production are modulated (increased and decreased) in a computer model one a time. Biosimulation is then conducted and the consequence of the modulation of a single biological process at different level of stimulation or inhibition is assessed by measuring clinical outcomes such as, but not restricted to, cartilage degradation rate, synovial cell density and IL-6 levels. The outcome of this analysis identified the biological processes that have significant impact on the clinical outcomes.

In the present invention, sensitivity analysis identified three areas of the biology of rheumatoid arthritis having a significant impact on disease pathophysiology: (1) monocyte/macrophage recruitment, (2) monocyte/macrophage apoptosis, and (3) macrophage cytokine (especially, TNF-α and IL-1) production.

1. Target Identification

We have discovered, based on the effects of galectin-3 activity inhibition by the model described above, blockade of galectin-3 activity is predicted to be an effective therapy for rheumatoid arthritis.

The effects of galectin-3 activity on monocyte/macrophage recruitment, macrophage apoptosis, and macrophage cytokine (particularly, TNF-α and IL-1) production were quantified and explicitly represented in a computer model of rheumatoid arthritis. As the contribution of galectin-3 activity on each of these biological processes is not precisely quantified, a range of effects was defined in order to characterize the contribution of galectin-3 activity (Table 1). The “lower max effect” value represents the lowest documented effect taking in consideration possible redundancies with other proteins, the “upper max effect” is the maximal effect of galectin-3 activity on each biological process and the “most likely max effect” is the estimation of the realistic contribution of galectin-3 activity in each biological process, taking in consideration the in vivo environment and potential redundancies with other proteins.

TABLE 1
Effect of galectin-3 Activity on Joint Model

| | Lower | Most likely | Upper |
|---|---|---|---|
| Hypothesis | max effect | max effect | max effect |
| monocyte recruitment | 0.65x | 0.5x | 0.4x |
| T-cell recruitment | 0.75x | 0.5x | 0.4x |
| monocyte/macrophage | 1x | 2x | 3x |
| apoptosis | | | |
| T-cell apoptosis | 1.5x | 2x | 3x |
| cytokine production | 0.87x | 0.75x | 0.65x |

Simulation of the effect of galectin-3 activity on rheumatoid arthritis was then conducted by blocking galectin-3 in all relevant biological processes at once or in one biological process at a time or in several biological processes in combination. The results of the simulation showed that blocking galectin-3 activity for 6 months could improve the rheumatoid arthritis clinical outcome by reducing synovial cell hyperplasia by 30 to 67%, cartilage degradation rate by 12 to 49%, and IL-6 levels in synovial tissue by 12 to 71%.

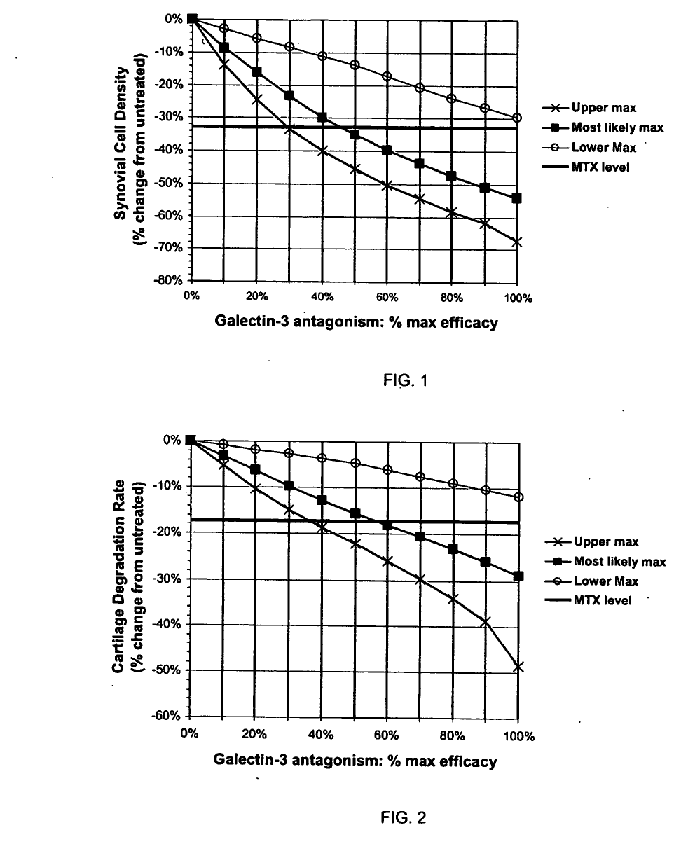

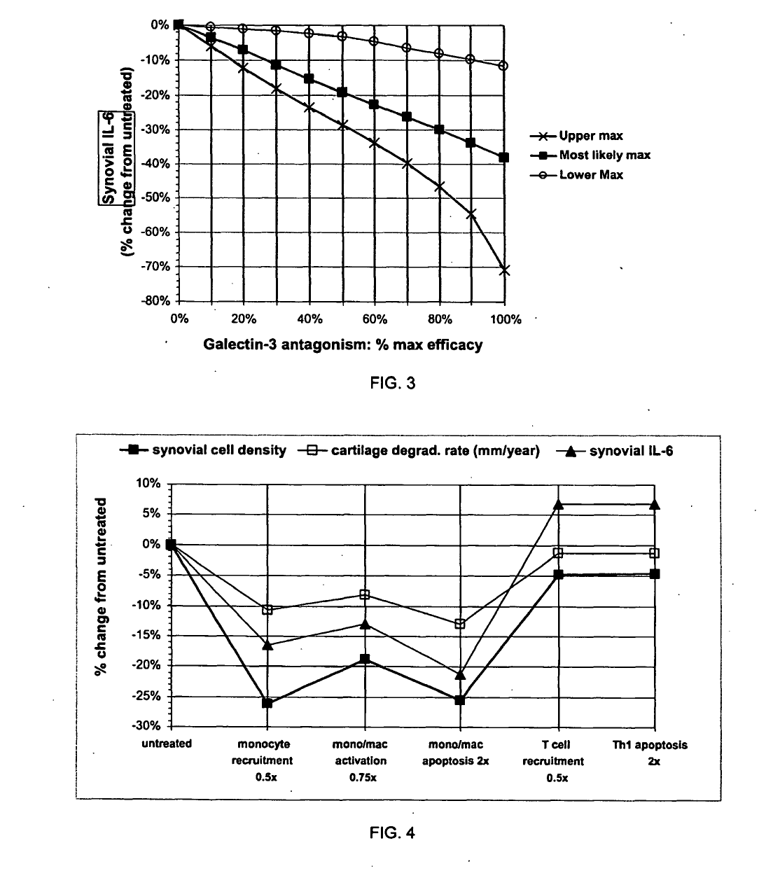

FIG. 1 demonstrates the effect of galectin-3 blockade on synovial cell density. To assess the length of time necessary to see the clinical impact of galectin-3 antagonist, the decrease in synovial cell density was determined at various time points during therapy over a six-month period. The effect on synovial cell density is near maximal after only 28 days and plateaus after 90 days (data not shown). This result indicates that an antagonist of galectin-3 activity is expected to produce a measurable clinical response after only two months of treatment. FIG. 2 demonstrates the effect of galectin-3 blockade on the rate of cartilage degradation. The effect of administration of a galectin-3 antagonist on cartilage degradation rate is near maximal after 28 days, similar to what was seen for synovial cell density. Again, this indicates that the effect of galectin-3 antagonist should give rapidly measurable clinical responses. FIG. 3 demonstrates the effect of galectin-3 blockade on IL-6 levels in synovial tissue.

Methotrexate is a common treatment for rheumatoid arthritis. Methotrexate treatment is known to decrease synovial cell density by approximately 30%, decrease the rate of cartilage degradation by approximately 15% and decrease the concentration of IL-6 in synovial tissue by 93%. At 100% efficacy, the computer model predicts galectin-3 antagonism is most likely to induce a greater improvement than methotrexate on synovial cell density and cartilage degradation rate. The model predicts that compounds causing only 60% inhibition of galectin-3 activity would be superior to methotrexate in decreasing the rate of cartilage degradation and would have a superior effect on the level of synovial cell density.

The simulation of galectin-3 blockade in one biological process at a time demonstrated that the main biological processes driving the impact of galectin-3 blockade on the clinical outcome are the effect on monocyte recruitment, macrophage apoptosis and macrophage activation (induction of macrophage cytokines). The impact of galectin-3 blockade on T-cell recruitment and apoptosis also plays a minor role in improvements in the clinical markers of rheumatoid arthritis. FIG. 4 provides the response of three key therapeutic indices in a typical rheumatoid arthritis patient upon simulation of galectin-3 blockade on monocyte recruitment, macrophage activation, macrophage apoptosis, T-cell recruitment, and T-cell apoptosis independently.

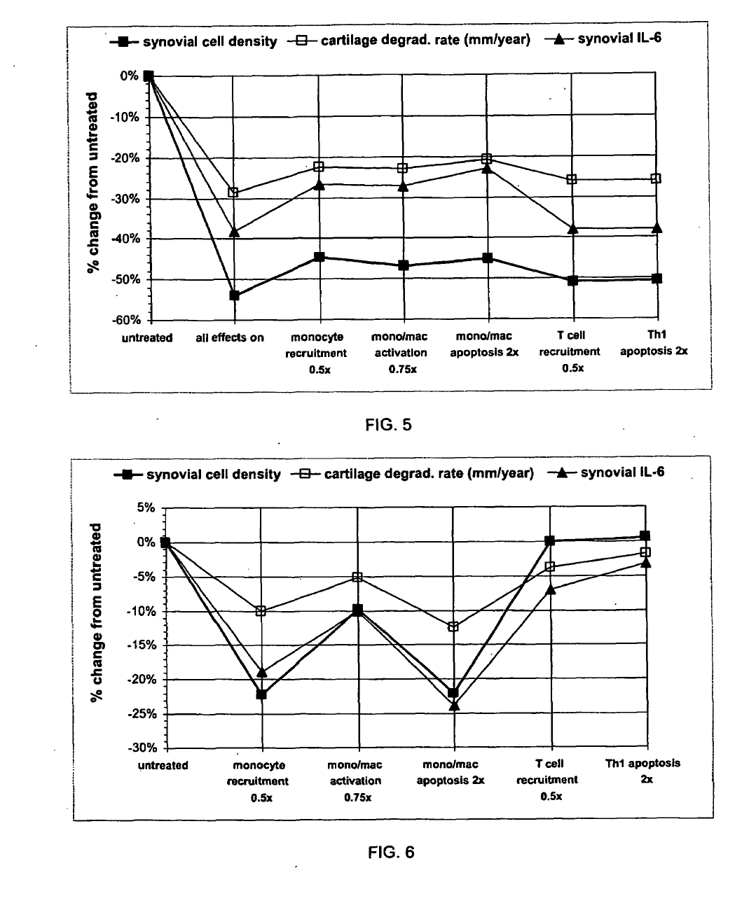

Conversely, by turning each effect off individually while leaving the other effects on, the potential synergic effects of different minor biological processes can be assessed (FIG. 5). At the most likely effect of galectin-3 blockade, there is significant redundancy of the effect on hyperplasia between the three major drivers (monocyte recruitment, monocyte/macrophage activation, and monocyte/macrophage apoptosis).

Some rheumatoid arthritis patients do not effectively respond to methotrexate treatment (initial non-responders), while other patients who initially responded to methotrexate become refractory over time (gradual non-responders). Both types of patients are referred to as methotrexate resistant patients. Simulation of blockading galectin-3 activity in a methotrexate resistant patient reveals a similar pattern of response than in a non-resistant patient. FIG. 6 illustrates the response of three key therapeutic indices in a typical rheumatoid arthritis patient upon simulation of galectin-3 blockade in a methotrexate resistant patient on monocyte and T-cell recruitment, monocyte/macrophage and T-cell apoptosis, and macrophage cytokine production. As in the standard rheumatoid arthritis patient, significant functional redundancy of galectin-3 antagonist effects occurs in the methotrexate-resistant patient (data not shown).

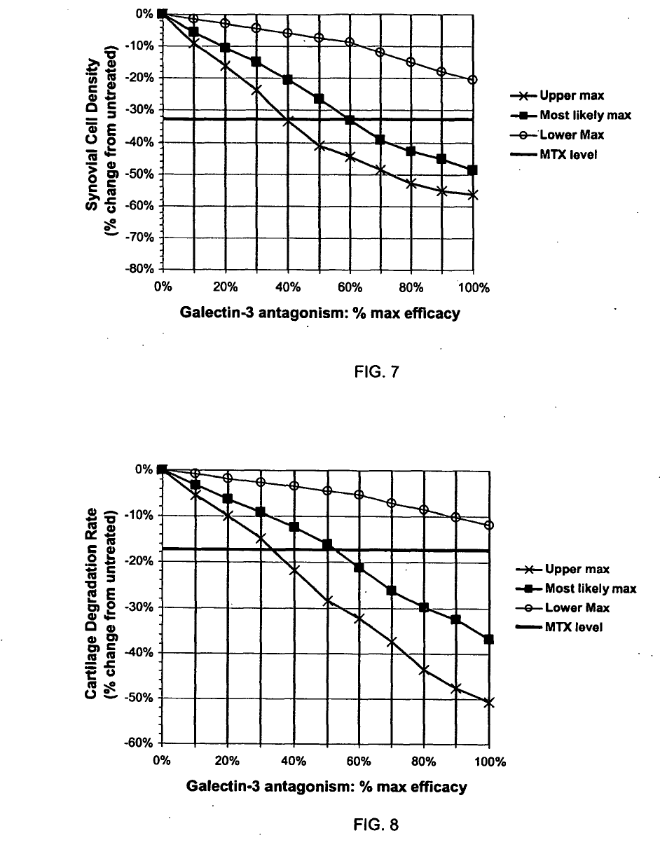

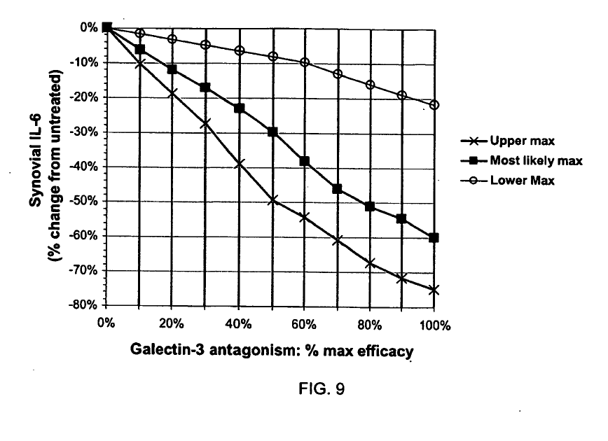

The results of the simulation showed that blocking galectin-3 activity for 6 months in a methotrexate resistant patient could improve the rheumatoid arthritis clinical outcome by reducing synovial cell hyperplasia by 21 to 56%, cartilage degradation rate by 12 to 51%, and IL-6 concentration by 22 to 75%. FIG. 7 demonstrates the effect of galectin-3 blockade on synovial cell density in a methotrexate resistant patient. FIG. 8 demonstrates the effect of galectin-3 blockade on cartilage degradation rate in a methotrexate resistant patient. FIG. 9 demonstrates the effect of galectin-3 blockade on IL-6 concentration in a methotrexate resistant patient.

Application of the in silico model of rheumatoid arthritis indicated that antagonism of galectin-3 activity is a promising therapeutic strategy for patients suffering from rheumatoid arthritis.

2. Thresholds

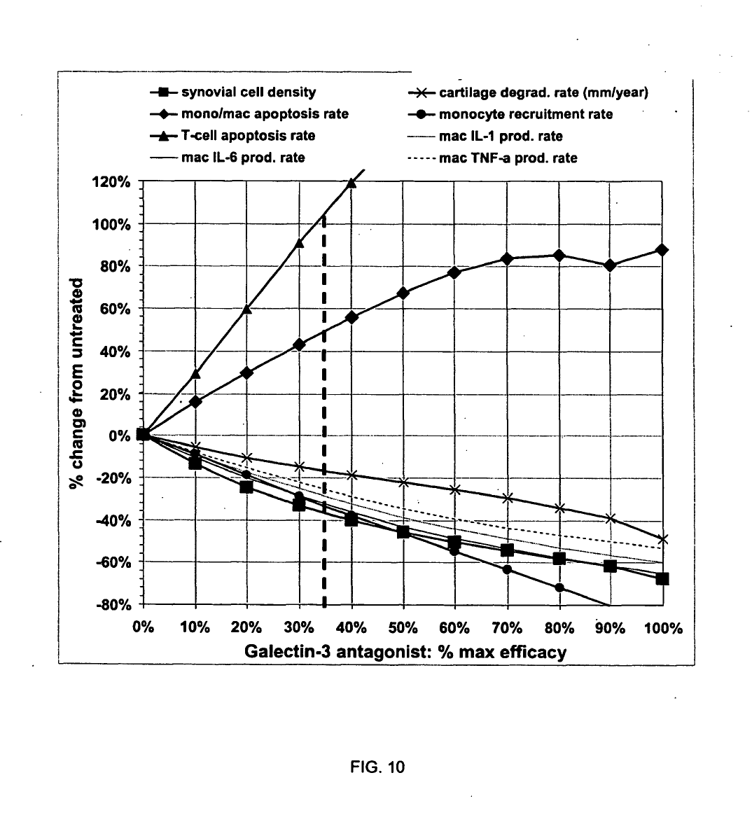

Although the amount of galectin-3 inhibition is correlated to decreased monocyte/macrophage recruitment, cytokine production and increased macrophage apoptosis, the alterations in recruitment rate, cytokine production and apoptosis rate are not linearly related to galectin-3 inhibition. FIG. 10 provides a comparison of galectin-3 inhibition with expected increase of macrophage apoptosis and decreased monocyte recruitment rates at the ‘upper maximum effect’ of galectin-3 antagonism. Each of these rates is compared to the therapeutic index of synovial cell density. The model showed that to achieve a significant improvement in rheumatoid arthritis symptoms (i.e., at least 30% decrease in synovial cell density) in the reference patient, macrophage apoptosis must increase by at least approximately 50% and the rate of monocyte recruitment must decrease by at least approximately 35% after 24 hours of galectin-3 blockade with a 30% decrease in cytokine production rate after one week of galectin-3 blockade.

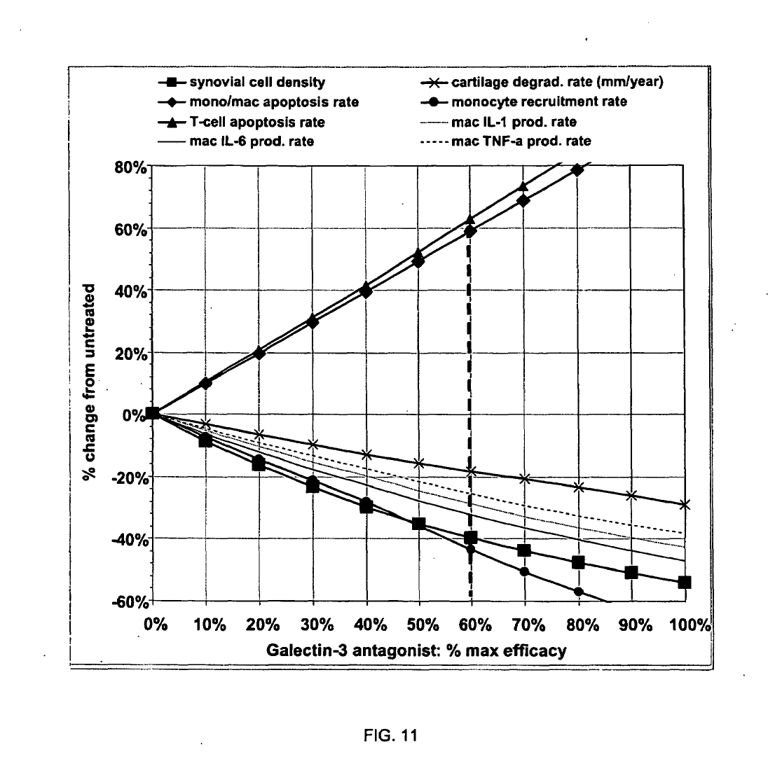

FIG. 11 provides a comparison of galectin-3 inhibition with expected increase of macrophage apoptosis and decreased monocyte recruitment and cytokine production rates at the ‘most likely maximum effect’ of galectin-3 antagonism. Each of these rates is compared to the therapeutic index of synovial cell density. The model found that to achieve a significant improvement in rheumatoid arthritis symptoms (i.e., at least 30% decrease in synovial cell density) in the reference patient, macrophage apoptosis must increase by at least approximately 60% and the rate of monocyte recruitment must decrease by at least approximately 45% after 24 hours of galectin-3 blockade with a 30% decrease in cytokine production rate after one week of galectin-3 blockade. In view of both hypotheses, the global threshold for therapeutic antagonism of galectin-3 activity would result in at least a 60% increase in macrophage apoptosis and in at least a 40% decrease in monocyte recruitment to the synovium.

D. Galectin-3

Galectin-3 is a β-galactoside-binding lectin that has both intracellular effects (anti-apoptotic, macrophage differentiation) and extracellular functions (chemokinetic/chemotactic factor) that are relevant to the physiopathology of rheumatoid arthritis. Galectin-3 is also known as MAC2 (macrophage galactose-specific lectin-2), L-29, CBP-35 (carbohydrate binding protein-35), and human IgE binding factor epsilon (epsilon BP).

Galectin-3 is evolutionarily highly conserved and has been shown to bind to purified laminin, thus possibly playing a role in the interaction between macrophages and the extracellular matrix. Galectin-3 is chemotactic at high concentrations but chemokinetic at low concentrations for human monocytes. In addition, galectin-3 causes calcium influx at high, but not low, concentrations. Cultured human macrophages and alveolar macrophages also respond to galectin-3. These effects are mediated, at least in part, by a pertussis-toxin sensitive receptor and not by receptors for chemokines, including CCR1, CCR2, CCR5, and CXCR4.

Galectin-3 is expressed in various tissues and organs. However, it does not seem to be expressed by normal hepatocytes, even though high levels of galectin-3 expression exist in most Hepatocellular carcinomas. Thus, galectin-3 expression has been suggested to be involved in tumor transformation and invasiveness, and may assist in tumor cell survival. While galectin-3 and its binding-protein have also been shown to be over-expressed in the rheumatic joint, the scientific community has not correlated blockade of galectin-3 activity with therapeutic benefit in rheumatoid arthritis.

E. Methods of Identifying Galectin-3 Antagonists and Anti-Rheumatic Drugs

1. Monocyte Recruitment

As described above, inhibiting monocyte/macrophage recruitment is a major contributor to the benefits of galectin-3 blockade. One preferred assay for identifying antagonists of galectin-3 activity is a modification of a typical transmigration assay. Monocytes are in suspension above an endothelial layer growing on a porous support above a lower well of endogenous (made by the endothelium) or exogenous chemoattractant. The monocytes that end up in the lower chamber at the end of the assay are counted as transmigrated. Compounds that inhibit the activity of galectin-3 will decrease the number of cells that migrate across the endothelial layer.

In one preferred assay, endothelial cells are cultured on hydrated Type I collagen gels overlaid with fibronectin. Components of the culture medium penetrate into the porous gel. Alternatively, the endothelial cells may be grown on the upper surface of a porous filter suspended above a lower chamber. Culture medium is placed in the upper and lower chambers to reach the apical and basal surfaces of the monolayer. Monocytes are added to the upper chamber. In order to be counted as “migrated”, a monocyte must (1) attach to the apical surface of the endothelial cells, (2) migrate to the intercellular junction, (3) diapedese between the endothelial cells, (4) detach from the endothelial cells and penetrate the basal lamina, (5) cross the filter or gel and (6) detach from the filter or gel and enter the lower chamber.

Monocytes, freshly isolated from peripheral blood of healthy or rheumatic donors are allowed to settle on confluent endothelial monolayers at 37° C. in the presence or absence of test compounds. The assays may be run in a variety of media including, but not limited to, complete medium, Medium 199, or RPMI1640, optionally supplemented with human serum albumin. After sufficient time for transendothelial migration, generally one hour, the monolayers are washed with a chelator, such as EGTA, to remove any monocytes or neutrophils still attached to the apical surface. If a collagen gel is used as a substrate, the monolayer is then rinsed with phosphate buffered saline with divalent cations and fixed in glutaraldehyde overnight. Fixing strengthens the collagen gel so that it is easier to manipulate. The monolayers are stained, preferably with Wright-Giemsa, and mounted on slides for direct observation, preferably under Nomarski optics. Using Nomarski optics, one can distinguish by the plane of focus, monocytes that are attached to the apical surface of the monolayer from those that have transmigrated. A quantifiable measure of transmigration is the percentage of those monocytes associated with the monolayer that have migrated through the monolayer. Therefore, the measurement of transmigration is independent of the degree of adhesion to the monolayer.

Migration of monocytes can be determined in the presence or absence of cytokine stimulation of the endothelium. Activation of endothelial cells can result from contact with stimulatory mediators and typically will enhance migration of monocytes or neutrophils across the endothelium. For the purpose of the present invention, activation of endothelial cells preferably results from contact with cytokines such as tumor necrosis factor (TNF) and interleukin-1 (IL-1).

The term “endothelial cell” has ordinary meaning in the art. Endothelial cells make up endothelium, which is found, inter alia, in the lumen of vascular tissue (veins, arteries, and capillaries) throughout the body. In arthritis leukocytes migrate from the circulating blood to the arthritic joint where they participate in inflammation.

2. Monocyte/Macrophage and T-Cell Apoptosis

As described above, inhibiting monocyte/macrophage apoptosis is the second major contributor to the expected benefits of galectin-3 blockade. Apoptosis measurement can vary depending on the cell type and the assay used. It may be advantageous to use a combination of standard apoptotic assays (e.g., Annexin V or TUNEL assays) to measure the percentage of apoptotic monocytes/macrophages and a quantitative anti-histone ELISA to measure the global effect of galectin-3 blockade on apoptosis.

a. DNA Fragmentation Assays

Loss of DNA integrity is a characteristic of apoptosis. When DNA extracted from apoptotic cells is analyzed using gel electrophoresis, a characteristic “ladder” of DNA fragments is seen. However, extraction of DNA from cells is a time consuming process and alternative methods are equally suitable for detecting the characteristic fragmentation of DNA in apoptotic cells. DNA fragmentation can be detected by a variety of assay including propidium iodide assays, acridine orange/ethidium bromide double staining, TUNEL and ISNT techniques, and assays of DNA sensitivity to denaturation.

b. Annexin V Assays

Externalization of phosphatidylserine (PS) and phosphatidylethanolamine is a hallmark of the changes in the cell surface during apoptosis. Annexin V is a 35-36 kDa Ca²⁺-dependent, phospholipid binding protein that has a high affinity for PS and binds to cells with exposed PS. Annexin V may be conjugated to any of a variety of markers to permit it to be detected by microscopy or flow cytometry. For use in methods of identifying compounds that inhibit galectin-3 activity or methods of screening for compounds that inhibit galectin-3 activity, it is preferable to use fluorescently labeled annexin V detected by flow cytometry.

Macrophages are obtained as discussed above from either rheumatoid or healthy subjects. Cells are incubated with the test compound for one to 24 hours, optionally in the presence of a death receptor-dependent inducer of apoptosis. The number of cells committed to apoptosis is determined by staining with labeled annexin V and a vital dye, such as propidium iodide (PI) or 7-amino-actinomycin D (7-AAD). Because externalization of PS occurs in the earlier stages of apoptosis, annexin V staining precedes the loss of membrane integrity which accompanies the latest stages of cell death resulting from either apoptotic or necrotic processes. Therefore, staining with annexin V in conjunction with vital dyes such as propidium iodide (PI) or 7-amino-actinomycin D (7-AAD) permits identification of early apoptotic cells (annexin V-positive and vital dye-negative).

3. Macrophage Activation

Another method for evaluating antagonists of galectin-3 activity is through the use of a macrophage activation assay. Macrophage activation can be assessed by measuring the production of cytokine after stimulation of the macrophage with lipopolysaccharide (100 ng/ml) or purified protein derivative of tuberculin (PPD) (10 μg/ml). Culture supernatants are then harvested and cytokine concentration measured using a method such as, but not limited to, sandwich ELISA.

The inhibition of macrophage activation (as measured by cytokine release) can be assessed by incubating a population of macrophages with both a stimulatory compound and an antagonist of galectin-3 activity (an antibody or other molecule). The residual cytokine production of the purified macrophages can be assessed using the assay previously described.

4. Galectin-3 Expression

The activity of galectin-3 can be antagonized by decreasing the expression of galectin-3 or by increasing the proteolytic degradation of expressed galectin-3. Methods of determining expression levels of proteins are well known in the art. Any measurement technique, now known or discovered in the future, may be used to determine the amount of galectin-3 protein that is expressed or present in a cell. The method exemplified herein it just one of the many acceptable methods for determining galectin-3 expression levels.

Monocytes or macrophages can be isolated from synovial fluid or peripheral blood mononuclear cells from rheumatoid arthritis patients or healthy donors by either Percoll or Histopaque (Sigma Chemical Co.) gradient centrifugation or countercurrent centrifugal elutriation (Beckman-Coulter). Monocytes can be differentiated into macrophages with RPMI containing 20% heat-inactivated fetal bovine serum (FBS) plus 1 μg/ml polymyxin B sulfate (Sigma Chemical Co.) in 24-well plates (Costar). The macrophages are incubated with a compound of the invention for periods of time ranging from one hour to several days. After incubation, the cells are lysed by any suitable method to produce a cell lysate. The amount galectin-3 expression can be determined via Western Blot, immunoprecipitation or any other quantitative procedure utilizing anti-galectin-3 antibodies. Suitable anti-galectin-3 antibodies include polyclonal and monoclonal antibodies. Any antibody or antibody fragment, polyclonal or monoclonal antibody specific for galectin-3 may be used to quantify galectin-3 protein levels. Appropriate negative controls, including cells treated identically to the test cells with the exception of exposure to the test compound should be performed in order to identify alterations in galectin-3 expression due to exposure to the compound rather than manipulations of the cells during experimentation.

Various procedures, well known in the art, may be used for the production of polyclonal antibodies to galectin-3. For example, for the production of polyclonal antibodies, various host animals, including but not limited to rabbits, mice, rats, etc., can be immunized by injection with galectin-3 or a derivative thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

A monoclonal antibody (mAb) to galectin-3 can be prepared by using any technique known in the art which provides for the production of antibody molecules by continuous cell lines in culture. These include but are not limited to, the hybridoma technique originally described by Kohler and Milstein (Nature 256:495-497 (1979)), the more recent human B cell hybridoma technique (Kozbor et al., Immunology Today 4:72 (1983)), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA and, IgD and any subclass thereof. The hybridoma producing the mAbs of use in this invention may be cultivated in vitro or in vivo.

F. Methods of Treatment

In one aspect, the invention provides methods of alleviating at least one symptom of an inflammatory disease, such as rheumatoid arthritis, comprising administering a therapeutically effective amount of an antagonist of galectin-3 activity to a patient having an inflammatory disease. The invention also provides methods for alleviating at least one symptom of rheumatoid arthritis comprising administering a therapeutically effective amount of an antagonist of galectin-3 activity to a patient having rheumatoid arthritis. The antagonist of galectin-3 activity maybe a protein, nucleic acid or small molecule inhibitor. A preferred protein antagonist is an antibody, more preferably a monoclonal antibody. Preferred nucleic acid antagonists include antisense inhibitors of the gene encoding galectin-3. The invention also encompasses methods of decreasing synovial cell density, methods of decreasing cartilage degradation and methods of decreasing IL-6 concentration in synovial tissue by administering a therapeutically effective amount of an antagonist of galectin-3 activity.

Antisense inhibitors have been shown to be capable of interfering with expression of target proteins. See Cohen, “Designing antisense oligonucleotides as pharmaceutical agents,” Trends Pharmacol Sci. 10:435-7 (1989) and Weintraub, “Antisense RNA and DNA,” Sci Am. 262:40-6 (1990), both incorporated herein by reference.

A compound useful in this invention is administered to a rheumatoid arthritis patient in a therapeutically effective dose by a medically acceptable route of administration such as orally, parenterally (e.g., intramuscularly, intravenously, subcutaneously, intraperitoneally), transdermally, rectally, by inhalation and the like. The dosage range adopted will depend on the route of administration and on the age, weight and condition of the patient being treated.

Various delivery systems are known and can be used to administer a composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into a rheumatic joint by any suitable route. Pulmonary administration can also be employed, e.g., by use of an inhaler or nebulizer, and formulation with an aerosolizing agent.

G. Combination Therapies

In one aspect, the invention provides methods of alleviating at least one symptom of rheumatoid arthritis, comprising administering an antagonist of galectin-3 activity and an anti-inflammatory drug to a patient having rheumatoid arthritis. Preferably, the anti-inflammatory drug is selected from the group of methotrexate, a TNF-α antagonist, an interleukin-1 receptor antagonist and a steroid. More preferably, the anti-inflammatory drug is methotrexate, Anakinra or prednisone. In one embodiment of the invention, the patient is resistant to methotrexate or to TNF-α blockade.

Various treatment protocols were simulated alone, or in combination with antagonism of galectin-3 activity. The effects of several therapies are represented in the model. The model reproduces the impact of treatment with (1) non-steroidal anti-inflammatory drugs (NSAIDs; e.g., indomethacin), (2) Etanercept, a soluble type II TNF-α receptor, (3) methotrexate (MTX), (4) glucocorticoids (e.g., methylprednisolone), and (5) Anakinra, an IL-1 receptor antagonist (IL-1Ra).

Each therapy is implemented based on its mode of action, molecular activity, and pharmacokinetic properties as well as its recommended clinical dosing regimen. To determine the importance of time-dependent variation in drug exposure associated with the clinically recommended periodic drug administration, we compared simulation results based on the clinical schedule with results for a constant-concentration continuous dose with an equivalent serum area-under-the-curve (AUC) net drug exposure. Simulation results for the two different administration schedules differed only minimally. In order to simplify presentation of results by eliminating transient effects due to periodic administration, results discussed herein are based on continuous dose therapy simulations.

The impact of the simulated treatments results from the implemented molecular activity. For example, Etanercept is modeled as binding and neutralizing TNF-α; any subsequent changes in hyperplasia, cartilage degradation, or other measurements are a secondary consequence of this reduction in free, active TNF-α, rather than a direct or specified effect of Etanercept. The effects directly implemented for each therapy are as follows:

The primary, common mode of action of NSAIDs is the inhibition of the cyclo-oxygenase (COX) pathways and synthesis of their downstream products, especially prostaglandin-E2 (PGE2). The model implementation of NSAIDs is based on in vitro data on the dose-dependent inhibition by NSAIDs of PGE2 synthesis in macrophages, FLS, and chondrocytes. Simulation results presented are for a constant continuous dose with serum AUC drug exposure equivalent to that achieved with a dosing schedule of 50 mg indomethacin, administered orally 3 times a day.

Etanercept and Infliximab (exogenous sTNF-R11 and anti-TNF-α antibody respectively) are modeled as binding and neutralizing soluble TNF-α. The binding of these agents to TNF-α is modeled using appropriate values for binding rate parameters of each molecule. The net binding rate of soluble receptors (or anti-TNF-α) to TNF-α is calculated as the difference between the binding and dissociation rates as follows:

$$\frac{d}{d t} [ TNF α \text{:} sTNFR ] = {k}_{on} [ TNF α ] [ sTNFR ] - {k}_{off} [ TNF α \text{:} sTNFR ] \qquad ( eq . 1 )$$

where
kₒₙ=constant of association between sTNF-R and TNF-α
k_{off}=constant of dissociation between sTNF-R and TNF-α
[TNFα]=concentration of free TNF-α
[sTNFR]=concentration of free soluble TNF-R
[TNFα:sTNFR]=concentration of bound complexes

Simulation results presented are for a constant continuous dose of Etanercept with serum AUC drug exposure equivalent to that achieved with a dosing schedule of 25 mg, administered subcutaneously twice a week.

Methotrexate therapy is implemented based on in vitro data that quantify its direct effects on particular cellular functions, including dose-dependent inhibition of T cell and FLS proliferation, mediator synthesis, and apoptosis. In addition, to account for the inhibitory effect of methotrexate on vascular proliferation and vascularization, a reduction in total endothelial adhesion molecules expression is also implemented. Simulation results presented are for a constant continuous dose with serum AUC drug exposure equivalent to a multiple of a dosing schedule of 12.5 mg/week, administered orally to account for long-lived, active metabolites of methotrexate.

Methylprednisolone is represented by the dose-dependent modulation of various cellular mediator synthesis rates according to in vitro data. Effects on other cell functions are not directly modeled but may arise from altered mediator-dependent regulation. Simulation results presented are for a constant continuous dose with serum AUC drug exposure equivalent to that of a dosing schedule of 5 mg methylprednisolone, administered orally once a day.

Anakinra, like endogenous IL-1Ra, is modeled as reducing the impact of IL-1β on all cellular functions. This is implemented by calculating an “effective” IL-1β concentration that has been adjusted to account for the impact of reduced receptor binding in the presence of the instantaneous concentration of receptor antagonist. Simulation results presented are for a constant continuous dose with serum AUC drug exposure equivalent to that of a dosing schedule of 100 mg Anakinra, administered subcutaneously once a day.

Simulation of the effect of treatment on the progression of rheumatoid disease in a virtual patient was conducted by simulating rheumatoid arthritis in the virtual patient for one year without treatment to establish a baseline in the model. Then either no treatment, a current treatment protocol or a current protocol in combination with galectin-3 antagonism was modeled. galectin-3 antagonism was modeled assuming 100% inhibition of galectin-3 activity having (i) the “upper max effect,” which represent maximal expected effect of galectin-3 activity on each biological process (ii) the “most likely max effect,” which is the estimation of the realistic contribution of galectin-3 activity, taking into consideration the in vivo environment and redundancies; and (iii) the “lower max effect,” which represents the lowest documented effect taking in consideration possible redundancies with other proteins. The effects of the simulated treatment (or lack of treatment) in a typical patient for six months on synovial cell density and cartilage degradation rate are shown in TABLE 2.

TABLE 2
Effects of Galectin-3 Antagonism in Combination with Other Therapies

| | | Reference patient | | MTX resistant patient | | TNF nonresponder | |
|---|---|---|---|---|---|---|---|
| First agent | Second agent | s.c.d. | c.d.r. | s.c.d. | c.d.r. | s.c.d. | c.d.r. |
| None | None | 100 | 100 | 100 | 100 | 100 | 100 |
| | anti-galectin-3 (lower maximum effect) | 70 | 88 | 79 | 88 | 72 | 88 |
| | anti-galectin-3 (most likely maximum effect) | 46 | 71 | 51 | 63 | 47 | 70 |
| | anti-galectin-3 (upper maximum effect) | 33 | 51 | 44 | 49 | 36 | 54 |
| NSAID | None | 103 | 105 | 105 | 106 | 104 | 106 |
| | anti-galectin-3 (lower maximum effect) | 76 | 94 | 89 | 94 | 77 | 94 |
| | anti-galectin-3 (most likely maximum effect) | 48 | 73 | 57 | 67 | 49 | 73 |
| | anti-galectin-3 (upper maximum effect) | 35 | 56 | 47 | 51 | 37 | 56 |
| Methotrexate | None | 67 | 82 | 81 | 87 | 70 | 83 |
| | anti-galectin-3 (lower maximum effect) | 50 | 72 | 59 | 72 | 52 | 72 |
| | anti-galectin-3 (most likely maximum effect) | 36 | 55 | 42 | 45 | 37 | 55 |
| | anti-galectin-3 (upper maximum effect) | 27 | 37 | 39 | 35 | 27 | 35 |
| Etanercept | None | 51 | 67 | 71 | 81 | 88 | 76 |
| | anti-galectin-3 (lower maximum effect) | 42 | 64 | 60 | 73 | 51 | 66 |
| | anti-galectin-3 (most likely maximum effect) | 34 | 54 | 49 | 59 | 34 | 53 |
| | anti-galectin-3 (upper maximum effect) | 30 | 47 | 44 | 49 | 30 | 44 |
| Anakinra | None | 82 | 55 | 90 | 54 | 90 | 60 |
| | anti-galectin-3 (lower maximum effect) | 54 | 43 | 58 | 40 | 56 | 44 |
| | anti-galectin-3 (most likely maximum effect) | 34 | 31 | 43 | 31 | 36 | 32 |
| | anti-galectin-3 (upper maximum effect) | 28 | 24 | 42 | 29 | 28 | 23 |
| Steroid | None | 59 | 59 | 70 | 64 | 61 | 58 |
| | anti-galectin-3 (lower maximum effect) | 45 | 53 | 54 | 53 | 47 | 52 |
| | anti-galectin-3 (most likely maximum effect) | 30 | 36 | 44 | 41 | 30 | 34 |
| | anti-galectin-3 (upper maximum effect) | 27 | 28 | 40 | 35 | 27 | 26 |

s.c.d. = % of synovial cell density as compared to untreated patient
c.d.r. = % of cartilage degradation rate as compared to untreated patient

The results of the simulation in a typical rheumatoid arthritis patient showed that blocking galectin-3 activity in addition to administration of an interleukin-1 receptor antagonist, such as Anakinra, can improve the rheumatoid arthritis clinical outcome by reducing cartilage degradation by 57 to 76% and synovial cell hyperplasia by 46 to 72%. Similarly, treatment with galectin-3 inhibition in combination with methotrexate or a steroid, such as methylprednisolone, shows decreases in synovial cell hyperplasia and cartilage degradation that cannot be achieved with a monotherapy.

Simulation of galectin-3 antagonism in combination with standard anti-rheumatic treatments in a methotrexate resistant patient revealed a pattern of response that varied slightly from that in a normal methotrexate-responsive patient. The effects of the simulated treatment in a methotrexate resistant patient (summarized in Table 2) showed that blocking galectin-3 activity in addition to administration of an interleukin-1 receptor antagonist, such as Anakinra, improves the rheumatoid arthritis clinical outcome of synovial cell hyperplasia to a lesser extent than seen in a typical rheumatoid arthritis patient. However, the response to administration of Anakinra in combination with inhibition of galectin-3 activity still shows an improvement in clinical outcome, reducing cartilage degradation by 60 to 71% and synovial cell hyperplasia by 42 to 58%. Interestingly, a combination therapy comprising galectin-3 and administration of methotrexate to a methotrexate resistant patient can improve the rheumatoid arthritis clinical outcome by reducing cartilage degradation and synovial cell hyperplasia to a greater extent than achieved by galectin-3 antagonism or methotrexate treatment alone. As with a typical rheumatoid arthritis patient, treatment with galectin-3 antagonism in combination with a steroid, such as methylprednisolone, shows decreases in synovial cell hyperplasia and cartilage degradation that cannot be achieved with either monotherapy

TNF-α neutralizing therapies have become increasingly important in treating rheumatoid arthritis patients. However, roughly a third of all rheumatoid arthritis patients fail to achieve a clinically significant response to TNF-α neutralizing therapies. Three potential classes of TNF-α blockade resistant patients were defined in the model described above. Synovial hyperplasia and cartilage degradation are differentially affected when TNF-α varies within different ranges, leading to the identification of three nonresponder classes within the current model. Specifically, patients with low initial TNF-α activity show decreased synovial hyperplasia, but minimal reduction in cartilage degradation in response to TNF-α blockade (cartilage nonresponders, or CNRs), while patients with negligible initial TNF-α activity show poor response in both synovial hyperplasia and cartilage degradation (double nonresponders or DNRs). Alternatively, insufficient neutralization of TNF-α in patients with abnormally high or resistant levels of TNF-α activity yields improvement in cartilage degradation but poor response in hyperplasia (hyperplasia nonresponders or HNRs). Mechanistically, in patients with low levels of TNF-α, rheumatoid disease was perpetuated by increased activity of alternate macrophage activating pathways (e.g., CD40-ligation), reduced activity of anti-inflammatory cytokines (e.g., IL-10), and increased activity of degradation-promoting cytokines (e.g., IL-1β). Nonresponding patients also showed altered responses to other therapies such as IL-1Ra (data not shown).

Patients who fail to achieve a significant clinical response to TNF-α blockade represent a sizable subset of the rheumatoid arthritis population. Simulation of galectin-3 antagonism in combination with standard anti-rheumatic treatments in a TNF-α hyperplasia nonresponder revealed a slightly different pattern of response than in a normal TNF-α-responsive patient. The effects of the simulated treatment (or lack of treatment) in a TNF-α hyperplasia nonresponder for six months on synovial cell density and cartilage degradation is shown in Table 2. Combination of galectin-3 antagonism with methotrexate, IL-1Ra or steroid treatment can result in less synovial cell hyperplasia and lower cartilage degradation rates as compared to the monotherapy or galectin-3 antagonism alone. Interestingly, blocking galectin-3 activity in addition to administration of an TNF-α antagonist, such as Etanercept, improves the rheumatoid arthritis clinical outcome to a greater extent than seen with either monotherapy, reducing cartilage degradation by 34 to 56% and synovial cell hyperplasia by 49 to 70%.

An antagonist of galectin-3 activity and another anti-rheumatoid drug are administered concurrently. “Concurrent administration” and “concurrently administering” as used herein includes administering an antagonist of galectin-3 activity and another disease modifying anti-rheumatoid drug in admixture, such as, for example, in a pharmaceutical composition or in solution, or as separate compounds, such as, for example, separate pharmaceutical compositions or solutions administered consecutively, simultaneously, or at different times but not so distant in time such that the antagonist of galectin-3 activity and other disease modifying anti-rheumatoid drug cannot interact.

Regardless of the route of administration selected, the antagonist of galectin-3 activity and other anti-rheumatoid drug are formulated into pharmaceutically acceptable unit dosage forms by conventional methods known to the pharmaceutical art. An effective but nontoxic quantity of the antagonist of galectin-3 activity and other anti-rheumatoid drug are employed in the treatment. The antagonist of galectin-3 activity and other anti-rheumatoid drug may be concurrently administered enterally and/or parenterally in admixture or separately. Parenteral administration includes subcutaneous, intramuscular, intradermal, intravenous, injection directly into the joint and other administrative methods known in the art. Enteral administration includes tablets, sustained release tablets, enteric coated tablets, capsules, sustained release capsules, enteric coated capsules, pills, powders, granules, solutions, and the like.

H. Pharmaceutical Compositions

An aspect of the invention provides methods of manufacturing a drug useful for treating rheumatoid arthritis in a warm-blooded animal. The drug is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th} or 19^{th} Edition of Remington's Pharmaceutical Sciences, Published by the Mack Publishing Co., Easton, Pa. 18040.

Unit doses or multiple dose forms are contemplated, each offering advantages in certain clinical settings. The unit dose would contain a predetermined quantity of an antagonist of galectin-3 activity calculated to produce the desired effect(s) in the setting of treating rheumatoid arthritis. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of inflammatory disease.

A unit dose will contain a therapeutically effective amount sufficient to treat rheumatoid arthritis in a subject and may contain from about 1.0 to 1000 mg of compound, for example about 50 to 500 mg.

In a preferred embodiment, the drug of the invention is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The drug of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compound will preferably be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition.

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5.0 to about 50% by weight (% w) in dosage units weighing between 5 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93, which are incorporated herein by reference, for a fuller discussion.

The drug of the invention may be administered parenterally, e.g., intravenously, intramuscularly, intravenously, subcutaneously, or intraperitoneally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In a specific embodiment, it may be desirable to administer the compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the composition can be delivered in a controlled release, or sustained release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used in a controlled release system (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target (e.g., the brain, kidney, stomach, pancreas, and lung), thus requiring only a fraction of the systemic dose (see, e.g., Goodson, in Medical Applications of Controlled Release, supra, vol. 2, pp. 115-138 (1984)). Other controlled release systems are discussed in the review by Langer (1990).

In a specific embodiment where the drug of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The invention also encompasses pharmaceutical compositions comprising an antagonist of galectin-3 activity contained in a container and labeled with instructions for use of the composition in the treatment of rheumatoid arthritis. The kit can further comprise instructions for using dosage. Accordingly, the invention contemplates an article of manufacture comprising packaging material and, contained within the packaging material, a compound that decreases the activity of galectin-3, wherein the packaging material comprises a label or package insert indicating that said compound modulates the activity of galectin-3 and can be used for treating the symptoms of rheumatoid arthritis.

### EXAMPLES

The following examples are provided as a guide for a practitioner of ordinary skill in the art. The examples should not be construed as limiting the invention, as the examples merely provide specific methodology useful in understanding and practicing an embodiment of the invention.

### A. Example 1

### Monocyte/T-Cell Recruitment

Human PBLs are isolated from the citrate-anticoagulated whole blood of healthy donors or patients with rheumatoid arthritis by dextran sedimentation and density separation over Ficoll-Hypaque. The mononuclear cells are washed and further purified on nylon wool and by plastic adherence, as previously described (Carr 1996). The resulting PBLs (>90% CD3⁺ T lymphocytes) are cultured in LPS-free RPMI/10% FCS for 15-18 h before use. Memory and naive CD4⁺ T lymphocyte subsets (CD45RO⁺ and CD45RA⁺, respectively) are isolated by negative selection using magnetic cell separation (MACS, Miltenyi Biotec, Bergisch Gladbach, Germany), following the manufacturer's instructions. HUVECs are isolated from umbilical cord veins (Jaffe, 1973) and established as primary cultures in M199 containing 10% FCS, 8% pooled human serum, 50 μg/ml endothelial cell growth factor (Sigma-Aldrich), 10 U/ml porcine intestinal heparin (Sigma-Aldrich), and antibiotics. Experiments are done on cells at passage two cultured on hydrated Type I collagen gels (Muller 1989) in 96-well culture plates. In certain experiments TNF-α or IL-1β (10 ng/ml and 10 U/ml final concentrations, respectively) or diluted synovial fluid from healthy donors or patients with rheumatoid arthritis are added to the culture media for the final 4-24 h.

The migration of monocytes or T-cells through a layer of endothelial cells is measured. The details of this assay are described in Muller et al., J Exp Med 176:819-828 (1992) and Muller et al., J Exp Med 178:449-460 (1993). Transendothelial migration is quantitated by Namarski optics as described in Liao et al., J Exp Med 182:1337-1343 (1995) and Muller et al., J Exp Med 178:449-460 (1993). Leukocytes are added to confluent monolayers of HUVECs grown on hydrated collagen gels. After incubation (1 h), nonadherent cells are removed by washing and the remaining adherent and transmigrated cells are fixed in place on the endothelial monolayer by overnight incubation in 2.5% glutaraldehyde in 0.1 M sodium cacodylate buffer at pH 7.4. Multiple high-power fields are observed under microscope and scored. Transmigration data are expressed as the percentage of the total cells that remained with the monolayer that were below the endothelium.

Transendothelial migration is also quantitated on cross-sections of paraffin-embedded monolayers. These specimens are prepared by carefully removing replicate sample monolayers and placing the endothelial surfaces against each other with the collagen gel sides facing outward. This avoids mechanical dislodgement of cells during the embedding process. After substitution in wax, the specimens are bisected so that cuts through the specimen produce cross sections of four monolayer samples (two different portions of each of the two monolayers). Quantitation is performed on three levels of such specimens separated y at least 50 μm so that different areas of the specimen are sampled and the same cells are not counted twice.

### B. Example 2

### Role of Galectin-3 in Monocyte Extravasation

Peripheral blood mononuclear cells (PBMCs) were isolated from 6 healthy volunteer donors and 6 rheumatoid arthritis (RA) patients following informed consent and in accordance with protocols approved by the AMC Medical Ethics committee. Donor blood was diluted with cold PBS and PBMCs isolated by centrifugation over Ficoll. The PBMC layer was recovered and washed twice with PBS. Cells were counted, and monocytes isolated using a Dynal monocytes negative isolation kit as per the manufacturer's instructions. Monocytes were resuspended at 2×106 cells/ml in RPMI medium containing 0.5% fetal calf serum.

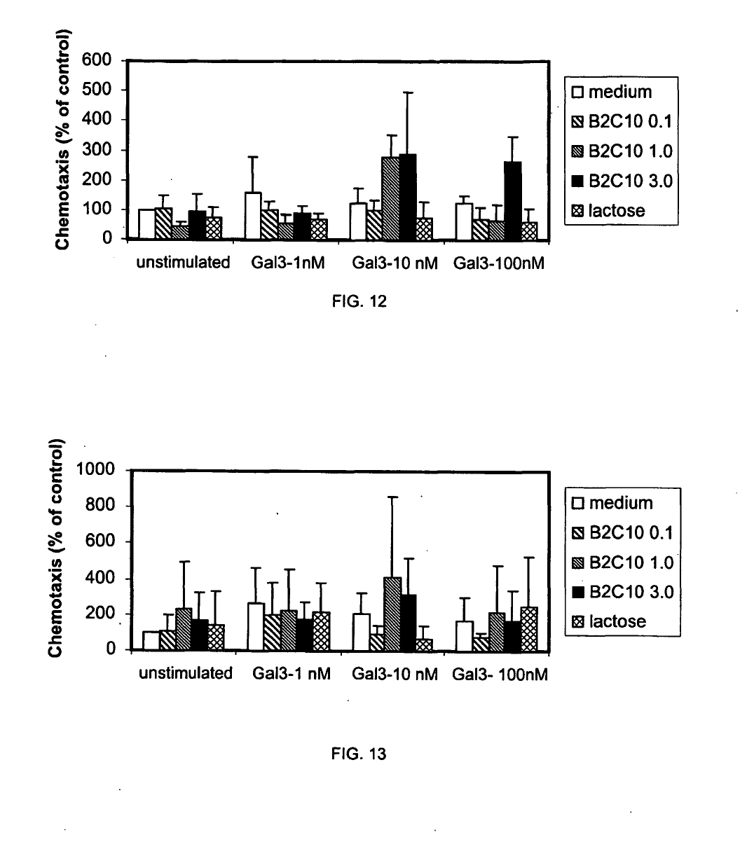

Chemotaxis assays were performed using 96-well disposable chemotaxis plates (Neuroprobe, 8 μm diameter pore size). Wells were preincubated for 1 hour at 37° C. with medium alone (RPMI containing 0.5% fetal calf serum), murine anti-human galectin-3 antibody B2C10 (0.1, 1 or 3 μg/ml) or lactose (10 mM). Lactose is a non-specific inhibitor of galectin-3. Purified recombinant human galectin-3 (1, 10 or 100 nM) was also included in the preincubation step. 5×10⁴ monocytes obtained from healthy donors were added to the top of the filter in triplicate wells and cells allowed to migrate for 90 minutes at 37° C. The filter was then washed with PBS, and adhering non-migrating cells removed from the top of the filter by wiping. Migrating cells within the filter were visualized by staining with crystal violet solution and washing with water. Following air-drying overnight, a computer digital imaging analyzer was used to capture 20 high-powered (400× magnification) fields encompassing the complete transwell filter, and migrating cells counted manually. FIG. 12 provides the relative amount migration (chemotaxis) in the presence of varying levels of galectin-3 and inhibitors of galectin-3 activity (B2C10 and lactose).

In a second experiment, wells were preincubated for 1 hour at 37° C. with medium alone (RPMI containing 0.5% fetal calf serum), murine anti-human galectin-3 antibody B2C10C (0.1, 1 or 3 μg/ml) or lactose (10 mM). Lactose is a non-specific inhibitor of galectin-3. Purified recombinant human galectin-3 (1, 10 or 100 nM) was also included in the preincubation step. 5×10⁴ monocytes obtained from rheumatoid arthritis patients were added to the top of the filter in triplicate wells and cells allowed to migrate for 90 minutes at 37° C. The filter was then processed as described above and migrating cells were counted manually. FIG. 13 provides the relative amount migration (chemotaxis) in the presence of varying levels of galectin-3 and inhibitors of galectin-3 activity (B2C10 and lactose).

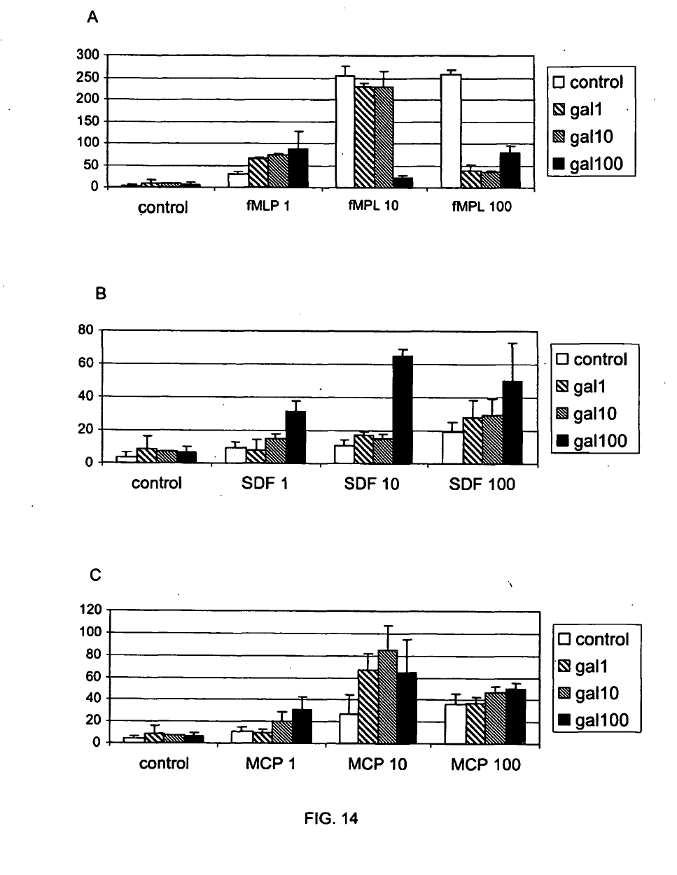

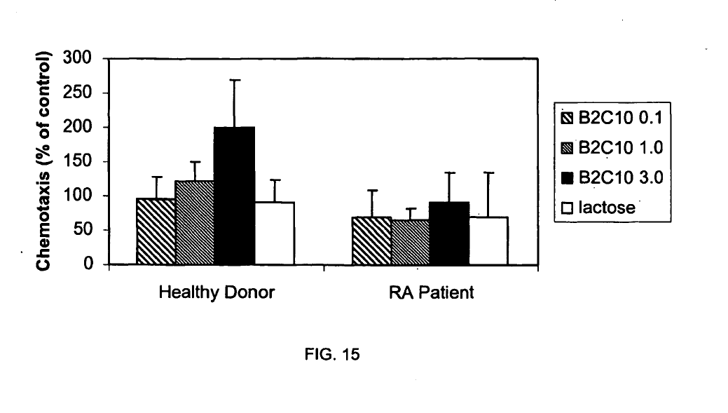

Galectin-3 alone does not reproducibly induce chemotactic activity in healthy donor or rheumatoid arthritis (RA) peripheral blood monocytes. However, in combination with other chemokines, galectin-3 can induce monocyte chemotaxis. wells were preincubated for 1 hour at 37° C. with medium alone (RPMI containing 0.5% fetal calf serum), medium containing N-formyl-met-leu-phe (fMLP) (1, 10 or 100 ng/ml), purified recombinant human galectin-3 (1, 10 or 100 nM), Stromal cell-Derived Factor-1 (SDF-1) (1, 10 or 100 nM), Monocyte Chemotactic Protein-1 (MCP-1) (1, 10, or 100 nM), or a 25% dilution of human RA patient synovial fluid (SF). Monocyte migration was assayed as described above. Galectin-3 in the presence of 1 ng/ml fMLP increases monocyte chemotaxis in a concentration dependent manner (FIG. 14A). SDF-1 (FIG. 14B) and MCP-1 (FIG. 14C) also induce migration of monocytes in combination with galectin-3. However, inclusion of the anti-galectin-3 antibody does not influence fMLP-induced chemotaxis of monocytes derived from healthy donor or rheumatoid arthritis patients (FIG. 15).

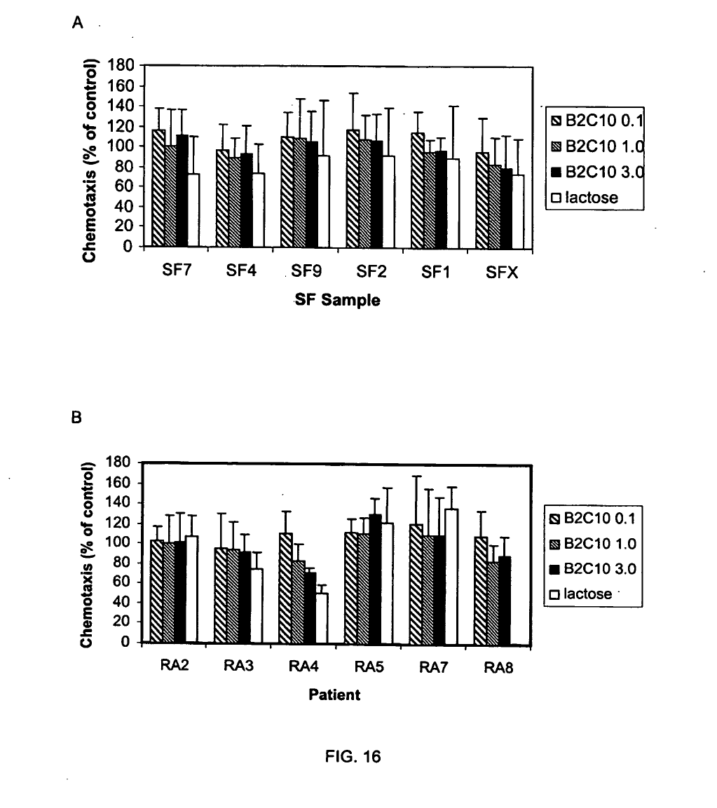

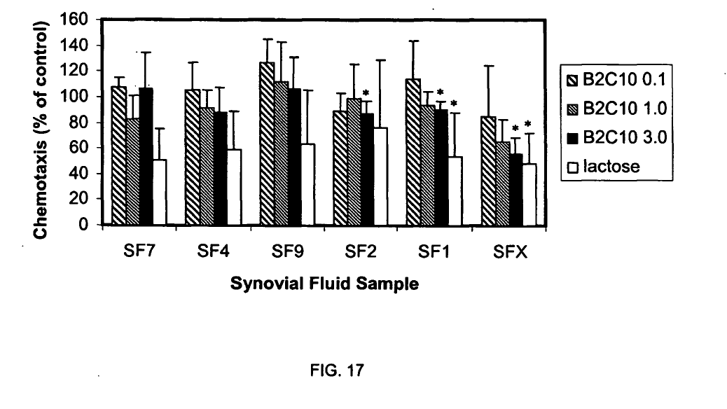

Monocytes from 6 rheumatoid arthritis patient were stimulated with 6 different synovial fluids and monocyte chemotaxis was measured as described above in the presence of different concentrations of galectin-3 blocking antibody. FIG. 16A illustrates the results of galectin-3 blockade across patients. FIG. 16B illustrates the results of galectin-3 blockade across the synovial fluid panel. The results show a trend toward concentration dependent inhibition of chemotaxis of synovial fluid-activated monocytes by the anti-galectin-3 antibody B2C10. However, due to the small sample size the apparent trend is not statistically significant. Repetition of the experiment with a large number of patients is expected to confirm inhibition of migration by inhibition of galectin-3 activity. Two patients (RA5 and RA7) demonstrate wide variation and no inhibition by anti-galectin-3 antibodies. If RA5 and RA7 are eliminated from the pool of patients, a statistically significant difference (indicated by asterisks) are observed for some synovial fluid (FIG. 17). Analysis of the differences in the composition of different synovial fluids may elucidate how inhibition of galectin-3 activity inhibits monocyte extravasation.

### C. Example 3

### Apoptosis Activation and Annexin V Assay

Isolated rheumatoid arthritis synovial fluid MNC and macrophages are incubated with 1 μg/ml of anti-Fas antibody (clone CH11; Beckman Coulter) or irrelevant IgM monoclonal antibody control for 24 hours in the presence or absence of the test compound. Cells are washed twice with cold PBS and then resuspended in 10 mM HEPES, pH 7.4; 140 mM NaCl; 2.5 mM CaCl₂ at a concentration of ˜1×10⁶ cells/ml. 100 μl of the solution (˜1×10⁵ cells) is transferred to a 5 ml culture tube. 5 μl of 2.5 μg Annexin V-phycoerythrin and 2.5 μg vital dye 7-AAD are added to each tube, gently mixed and incubated at room temperature in the dark for 15 minutes. 400 μl phosphate buffered saline (PBS) is added to each tube and the cells are analyzed by cell cytometry as soon as possible (within one hour). The percentage of apoptotic cells is measured by the percentage of Annexin V positive cells.

### D. Example 4

### TUNEL Assay

Apoptosis is induced in synovial MNC and macrophages by incubating the cells for 24 h with recombinant TNF-α (10 ng/ml), or 1 μg/ml anti-Fas, anti-TNF-R1 or anti-TRAIL receptors antibodies in the presence or absence of the test compound. 1-2×10⁶ monocytes are centrifuged at 400×G for minutes, the supernatant is discarded and the cells are resuspended in 0.5 ml phosphate buffered saline (PBS). The cells are fixed by adding the cell suspension to 5 ml of 1% (w/v) paraformaldehyde in PBS, placing it on ice for 15 min, washing the cells twice in PBS twice, and finally combining the cells suspended in 0.5 ml PBS with 5 ml ice-cold 70% (v/v) ethanol. The cells stand for a minimum of 30 minutes on ice or in the freezer before proceeding to the staining step.

The tubes are swirled to resuspend the cells and 1.0 ml aliquots of the cell suspensions (˜2-4×10⁵ cells/ml) are removed and placed in 12×75 mm centrifuge tubes. The cell suspensions are centrifuged for 5 min at 300×g and the 70% (v/v) ethanol removed by aspiration. The cells are washed twice by centrifugation and resuspension in PBS plus 0.05% sodium azide, pelleted and then resuspended in 50 μl Staining Solution (TdT enzyme/FITC-dUTP in cacodylate buffered saline). The cells are incubated at 37° C. for at least one hour. The staining is stopped by the addition of 1.0 ml PBS pus 0.05% sodium azide. The cells are pelleted by centrifugation at 300×g for 5 min, resuspended in PBS pus 0.05% sodium azide, and the repelleted. The supernatant is removed by aspiration and the pellet is incubated for 30 minutes at room temperature in the dark. The cells are analyzed by flow cytometry.

### E. Example 5

### Propidium Iodide Staining

Nine-day adherent synovial fluid macrophages are incubated with anti-Fas antibody or control IgM in the presence or absence of the test compound for 24 hours. Cultures are then harvested by 0.02% EDTA, fixing overnight in 70% ethanol, stained with propidium iodide (Roche Molecular Biochemicals, Indianapolis, Ind.), and the subdiploid peak, immediately next to the G₀/G₁ peak (2N), is determined by flow cytometry. It may be necessary to exclude objects with minimal light scatter, possibly representing debris, which would artificially increase the estimate of the subdiploid population. Typically, the percentage of apoptotic synovial macrophages (subdiploid population) increase from 2-5% in absence of an galectin-3 antagonist to 35-40% when galectin-3 activity is completely suppressed.

### F. Example 6

### Anti-Histone Sandwich Assay

Apoptosis is induced by incubating 104 synovial MNC or macrophages with 1 μg/ml anti-Fas antibody (CH11) or TNF-α (10 ng/ml) for 24 h in the presence or absence of the test compound. After the incubation, the cells are pelleted by centrifugation and the supernatant (containing DNA from necrotic cells that leaked through the membrane during incubation) is discarded. The cells are resuspended in Lysis Buffer and incubated 30 min at room temperature. After lysis, cell nuclei and unfragmented DNA are pelleted by centrifugation at 20 OOOx g for 10 min.

An aliquot of the supernatant (i.e., cytoplasmic fraction) is transferred to anti-histone antibody well of a microtiter plate. The complexes are bound to the plate via streptavidin-biotin interaction. The immobilized antibody-DNA-antibody complexes are washed three times to remove any components that are not immunoreactive. The bound complexes are detected with anti-DNA (peroxidase-conjugated) monoclonal antibodies revealed by a peroxidase substrate and amount of colored product (and thus, of immobilized antibody-histone complexes) is determined spectrophotometrically. The quantitative colorimetric measurement of the DNA-histone complex is proportional to the total amount of apoptotic cells present in the cell population tested.

### G. Example 7

### Macrophage Activation Assay

For quantitative measurement of secreted cytokines, 1×10⁶ macrophages are incubated with lipopolysaccharide (100 ng/ml) or PPD (10 μg/ml) for 12 to 24 h at 37° C./5% CO₂. Culture supernatants were then harvested and a sandwich ELISA assay is performed to measure the production of IL-1, and TNF-α using manufacturer's protocol (R&D Systems Inc., Minneapolis, Minn.).

### H. Example 8

### Western Blot Analysis of Galectin-3 Expression

Whole-cell extracts are prepared from synovial MNC and macrophages by lysis in 0.1% NP-40 lysis buffer. 25 to 50 μg of extract are analyzed by SDS-PAGE on 12.5% polyacrylamide gels, and transferred to ImmobilonP (Millipore) by semidry blotting. Filters are blocked for 1 h at room temperature in PBS/0.2% Tween-20/5% nonfat dry milk. Filters are blotted with rabbit anti-galectin-3 antibodies at 4° C. in PBS/0.2% Tween-20/2% nonfat dry milk. Filters are washed in PBS/0.2% Tween 20/2% nonfat dry milk and incubated with donkey anti-rabbit or anti-mouse secondary antibody (1:2,000 dilution) conjugated to horseradish peroxidase (Amersham PharmaciaBiotech). Visualization of the immunocomplex is performed using Enhanced Chemiluminescence Plus kit (AmershamPharmacia Biotech).

### I. Example 9

### Impact of Anti-Galectin-3 Monoclonal Antibody B2C10 in RA Mouse Model

Galectin-3 Ab was evaluated for possible anti-inflammatory activity in BALB/c mice with arthritis induced by monoclonal antibodies (mAb) raised against type II Collagen, plus lipopolysaccharide. Galectin-3 Ab (1, 0.5 and 0.25 mg/mouse×3) was administered intraperitoneally once daily for 3 consecutive days starting on day 3. Volumes of both hind paws were measured on days 5, 7, 10, 14 and 17 and the sum of both hind paw volumes was calculated. A 30% percent or more reduction (−30%) relative to the vehicle-treated group indicates significant.

Galectin-3 Ab, B2C10 from Pharmingen, was dissolved in distilled water. Test substance at 1, 0.5 and 0.25 mg/mouse and vehicle were each administered intraperitoneally once daily for 3 consecutive days starting on day 3. The dosing volume was 0.2 ml/mouse for the test compound or 10 ml/kg (PO) for indomethacin. All treatments are summarized below:

Inflammation, Collagen Arthritis, mAb in Mice

| | | | Conc. | | | |
|---|---|---|---|---|---|---|
| | Test | | (mg/ | | | Animals |
| Group | substances | Route | ml) | Dosage | | No. |
| 1 | Vehicle (PBS | IP | — | 0.2 | ml/mouse × 3 | 7 |
| | pH 7.4) | | | | | |
| 2 | Galectin-3 Ab | IP | 5 | 1 | mg/mouse × 3 | 7 |
| 3 | Galectin-3 Ab | IP | 2.5 | 0.5 | mg/mouse × 3 | 7 |
| 4 | Galectin-3 Ab | IP | 1.25 | 0.25 | mg/mouse × 3 | 7 |
| 5 | Indomethacin | IP | 0.3 | 3 | mg/kg | 7 |

BALB/c derived male mice, weighing 22±2 g were provided by National Laboratory Animals Breeding and Research Center (NLABRC, R. O. C.). The animals were housed in Individually Ventilated Cages Racks (IVC Racks, 36 Mini Isolator systems) under Specific Pathogen-Free (SPF) condition throughout the experiment. Each APEC cage was sterilized with autoclave and contained 7 mice (in cm, 26.7 length×20.7 width×14.0 height), and then maintained in a hygienic environment under controlled temperature (22°-24° C.) and humidity (50%-60%) with 12-hour light/dark cycle. The animals were given free access to sterilized lab. chow and sterilized distilled water ad libitum. All aspects of this work, i.e. housing, Experimentation and disposal of animals were performed in general accordance with the Guide for the Care and Use of Laboratory Animals (National Academy Press, Washington, D.C., 1996).

Collagen Arthritis, mAb Groups of 7 BALB/c strain mice, 6-7 weeks of age, were used for the induction of arthritis by monoclonal antibodies (mAbs) raised against type II collagen, plus lipopolysaccharide (LPS). A combination of 4 different mAbs (D8, F10, DI-2G and A2) totaling 4 mg/mouse was administered to the animals intravenously on day 0, followed by intravenous challenge with 25 mg/mouse of LPS 72 hours later (day3). From one hour after LPS injection, Galectin-3 Ab (1, 0.5 and 0.25 mg/mouse) and vehicle were each administered intraperitoneally once daily for 3 consecutive days starting on day 3. Whereas, indomethacin which served as the positive control was administered orally (3 mg/kg) once daily for 3 consecutive days at the same time schedule as test compound. For each animal, volumes of both hind paws were measured using a plethysmometer with water cell (12 mm diameter) on Days 0, 5, 7, 10, 14 and 17. Percent inhibition of increase in volume was calculated by the following formula:

Inhibition(%):[1−(Tn−T₀)/(Cn—C₀)]×100%

Where:

C₀ (Cn): volume of day 0 (day n) in vehicle control

T₀ (Tn): volume of day 0 (day n) in test compound-treated group.

Reduction of edema in the hind paws by 30% or more (³ 30%) is considered significant. In addition, body weight was documented during the testing period of day 0, 5, 7, 10, 14 and 17 in each animal percentage of body weight of each compound group at time points were calculated referring to the vehicle control group at each measurement day as 100%.

Administration of Galectin-3 Ab at 1 and 0.5 mg/mouse×3 caused non-significant reduction but moderate reduction of 22% and 26% on day 5, 27% and 16% on day 7, respectively. Concurrently tested indomethacin (3 mg/kg×3, PO) provided significant anti-inflammatory effects of 94%, 70%, 85%, 85% and 84% on days 5, 7, 10, 14 and 17, respectively, relative to the vehicle-treated group. There was no significant impact on the animal's weight. The results, with significant changes in parenthesis on days 5, 7, 10, 14 and 17 are summarized in the table below:

| | | | | (%) Reduction of Edema vs. Vehicle Control | | | | |
|---|---|---|---|---|---|---|---|---|
| Treatment | Route | Dose | | Day 5 | Day 7 | Day 10 | Day 14 | Day 17 |
| Galectin-3 Ab | IP | 1 | mg/mouse × 3 | 22 | 27 | 5 | −3 | −4 |
| | IP | 0.5 | mg/mouse × 3 | 26 | 16 | −3 | 6 | 2 |
| | IP | 0.25 | mg/mouse × 3 | 14 | 14 | −5 | 8 | −12 |
| Indomethacin | PO | 3 | mg/kg × 3 | (94) | (70) | (85) | (85) | (84) |

Inhibition of galectin-3 activity by administration of a blocking antibody decreases inflammation. The inhibition of inflammation dissipates shortly after administration of the blocking antibody was discontinued (after day 5) as expected. Optimization of dosage amounts and scheduling is expected to increase the anti-inflammatory effect of inhibition of galectin-3 activity to statistically significant levels.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1-24. (canceled)

25. The method of claim 55, wherein the amount of galectin-3 activity is measured by a process comprising the step of:
(a) comparing an amount of leukocytes that migrate through at least one layer of endothelial cells in the presence of the compound with an amount of leukocytes that migrate through at least one layer of endothelial cells in the absence of the compound; and
wherein the amount of leukocytes that migrate represents the amount galectin-3 activity.

26. The method of claim 25, wherein the compound is identified as useful in the treatment of rheumatoid arthritis when the amount of leukocytes that migrate in the presence of the compound is at least 35% lower than the amount of leukocytes that migrate in the absence of the compound.

27. The method of claim 26, wherein the compound is identified as useful in the treatment of rheumatoid arthritis when the amount of leukocytes that migrate in the presence of the compound is at least 60% lower than the amount of leukocytes that migrate in the absence of the compound.

28. The method of claim 27, wherein the compound is identified as useful in the treatment of rheumatoid arthritis when the amount of leukocytes that migrate in the presence of the compound is at least 95% lower than the amount of leukocytes that migrate in the absence of the compound.

29-33. (canceled)

34. The method of claim 55, wherein a decrease in galectin-3 activity in the presence of the compound is identified by observing an amount of leukocyte apoptosis in the presence of the compound that is higher than an amount of leukocyte apoptosis in the absence of the compound.

35. The method of claim 34, wherein the leukocytes are macrophages.

36. The method of claim 35, wherein the compound is identified as useful in the treatment of rheumatoid arthritis when the amount of macrophage apoptosis in the presence of the compound is at 50% greater than the amount of macrophage apoptosis in the absence of the compound.

37-38. (canceled)

39. The method of claim 35, wherein the amount of macrophage apoptosis is measured by a process comprising the steps of: (1) exposing a population of macrophages to an inducer of apoptosis in the presence or absence of the compound; and (2) measuring the percentage of cells in the population having DNA fragmentation wherein the percentage of cells having DNA fragmentation represents the amount of macrophage apoptosis.

40-43. (canceled)

44. The method of claim 35, wherein the amount of macrophage apoptosis is measured by a process comprising the steps of: (1) exposing a population of macrophages to an inducer of apoptosis in the presence or absence of the compound; and (2) measuring a percentage of macrophages in the population expressing phosphatidylserine on the extracellular surface of the cell membrane wherein the percentage of macrophages expressing phosphatidylserine on the extracellular surface of the cell membrane represents the amount of macrophage apoptosis.

45-46. (canceled)

47. The method of claim 44, wherein the percentage of macrophages expressing phosphatidylserine present on the extracellular surface of the cytoplasmic membrane is measured by binding of annexin V to the phosphatidylserine.

48. (canceled)

49. The method of claim 55, wherein a decrease in galectin-3 activity in the presence of the compound is identified by observing an amount of a cytokine produced by a first population of macrophages in the presence of the compound that is lower than an amount of the cytokine produced by a second population of macrophages in the absence of the compound.

50. (canceled)

51. The method of claim 49, wherein the compound is identified as useful in the treatment of rheumatoid arthritis when the amount of cytokine produced by the first population of macrophages in the presence of the compound is at least 40% lower than the amount of cytokine produced by the second population in the absence of the compound.

52. The method of claim 51, wherein the compound is identified as useful in the treatment of rheumatoid arthritis when the amount of cytokine produced by the first population of macrophages in the presence of the compound is at least 60% lower than the amount of cytokine produced by the second population in the absence of the compound.

53. The method of claim 52, wherein the compound is identified as useful in the treatment of rheumatoid arthritis when the amount of cytokine produced by the first population of macrophages in the presence of the compound is at least 80% lower than the amount of cytokine produced by the second population in the absence of the compound.

54. (canceled)

55. A method identifying a compound useful in the treatment of rheumatoid arthritis, which method comprises: (a) comparing an amount of galectin-3 activity in the presence of the compound with an amount of galectin-3 activity in the absence of the compound; and (b) selecting the compound as useful in the treatment of rheumatoid arthritis when the amount of galectin-3 activity in the presence of the compound is lower than the amount of galectin-3 activity in the absence of the compound.

56. The method of claim 55 for screening a collection of compounds, further comprising repeating steps (a) and (b) for each compound of the collection, wherein at least one compound of the collection is selected as useful for the treatment of rheumatoid arthritis.

57. The method of claim 55, wherein the compound is selected as useful in the treatment of rheumatoid arthritis when the amount of galectin-3 activity in the presence of the compound is at least 35% lower than the amount of galectin-3 activity in the absence of the compound.

58. The method of claim 57, wherein the compound is selected as useful in the treatment of rheumatoid arthritis when the amount of galectin-3 activity in the presence of the compound is at least 60% lower than the amount of galectin-3 activity in the absence of the compound.

59. The method of claim 58, wherein the compound is selected as useful in the treatment of rheumatoid arthritis when the amount of galectin-3 activity in the presence of the compound is at least 95% lower than the amount of galectin-3 activity in the absence of the compound.

60-61. (canceled)
